(19) United States
(12) Patent Application Publication
(10) Pub. No.: US 2024/0287173 A1
(43) Pub. Date: Aug. 29, 2024

(54) THERAPEUTIC AGENT FOR MUSCULAR ATROPHY

(71) Applicant: NATIONAL UNIVERSITY CORPORATION KUMAMOTO UNIVERSITY, Kumamoto (JP)

(72) Inventors: Yusuke ONO, Kumamoto (JP); Shin FUJIMAKI, Kumamoto (JP)

(73) Assignee: NATIONAL UNIVERSITY CORPORATION KUMAMOTO UNIVERSITY, Kumamoto (JP)

(21) Appl. No.: 18/552,609

(22) PCT Filed: Mar. 29, 2022
(86) PCT No.: PCT/JP2022/015725

§ 371 Date: May 10, 2024

(30) Foreign Application Priority Data
Mar. 30, 2021 (JP) 2021-056481

(51) Int. Cl. C07K 16/28; A61K 39/00; A61P 21/00; C07K 14/705; C12N 15/113

(52) U.S. Cl. CPC C07K 16/28; A61P 21/00; C07K 14/705; C12N 15/1138; A61K 2039/505; C07K 2317/76; C07K 2319/30; C12N 2310/14

(57) ABSTRACT

An object of the present invention is to provide a novel preventive or therapeutic agent for muscular atrophy. The present invention provides a preventive or therapeutic agent for muscular atrophy containing a Dll4 function inhibitor as an active ingredient. The Dll4 function inhibitor is selected from the group consisting of a binding inhibitor between Dll4 and Notch2, a Dll4 expression inhibitor, or an inhibitor of binding of collagen to LL4.

### TECHNICAL FIELD

The present invention relates to a therapeutic agent for muscular atrophy. More specifically, the present invention relates to a novel therapeutic agent for muscle atrophy containing a Dll4 function inhibitor as an active ingredient.

### BACKGROUND ART

Muscle atrophy is the atrophy of myofibers due to a decrease in myofibrils, resulting in pathological muscle weakness such as dyskinesia and mobility impairment. Diseases accompanied by muscle atrophy are roughly divided into two types, depending on their causes: neurogenic amyotrophic disease (amyotrophic diseases caused by motor nerve damage) and myogenic amyotrophic disease (amyotrophic diseases caused by damage to the muscle tissue itself).

Representative neurogenic amyotrophic diseases include amyotrophic lateral sclerosis (ALS), spinal muscular atrophy, spinobulbar muscular atrophy, Guillain-Barré syndrome and the like. At present, there are no effective treatments or therapeutic drugs for these diseases, and effective therapeutic agents are eagerly desired.

In addition, as myogenic amyotrophic diseases, there are non-hereditary muscle diseases such age-related muscular atrophy (sarcopenia), and disuse muscular atrophy caused by decreased activity due to illness or bedridden cases, and the like, in addition to genetic muscle diseases such as muscular dystrophy, congenital myopathy, distal myopathy and the like. In particular, age-related muscular atrophy and disuse muscular atrophy are on the rise with the aging of society. At present, there are no effective preventive drugs or therapeutic drugs for these various types of muscular atrophy, and there is an urgent need to develop effective preventive and therapeutic drugs.

Adult skeletal muscle is a highly plastic tissue that readily decreases or increases its mass in response to mechanical and metabolic stimuli. Notch which is a transmembrane protein has attracted attention as a molecule that controls the size of skeletal muscle. Notch is known to be composed of families 1-4, and to control fate decisions such as proliferation and differentiation in tissue stem cells including satellite cells (muscle satellite cells) in skeletal muscle.

The present inventors have compared the expression levels of Notch2 mRNA and protein in skeletal muscle of mice in which satellite cells were specifically killed and skeletal muscle of normal mice, and found that Notch2 was expressed not only in satellite cells but also in myofibers. It has been reported that transgenic mice (N2ICD-Tg mice) in which skeletal myofiber-specific active Notch2 was expressed constitutively were generated, and their phenotypes were analyzed, and muscle atrophy was observed in the fast muscles of N2ICD-Tg mice, while, there was no significant difference in the number of myofiber cell nuclei between N2ICD-Tg mice and control mice, and that constitutive activation of Notch2 signaling in myofibers has a negative effect on maintaining muscle mass (Non-Patent Document 1).

The present inventors have reported that Notch1 and Notch2 are essential for the maintenance of the quiescent and undifferentiated satellite cells, which are skeletal muscle stem cells (Non-Patent Document 2). In addition, the present inventors have reported that the inventors generated mice lacking both Notch1 and Notch2 in a myofiber-specific manner (Mlc1fCre/+:Notch1f/f:Notch2f/f) and induced muscle atrophy due to tail suspension and diabetes, then, muscle mass and myofiber cross-sectional area (CSA) were markedly decreased in wild-type mice, whereas resistance to muscle atrophy was observed in Notch1/Notch2-deficient mice (Non-Patent Document 3).

There are five known Notch ligands in mammals: Jagged1 (Jag1), Jagged2 (Jag2), Delta-like ligand-1 (Dll1), Delta-like ligand-3 (Dll3), and Delta-like ligand-4 (Dll4) (Non-Patent Document 4, Non-Patent Document 5). Among them, Dll1 and Dll4 have been reported to have similar structures, but have different functions (Non-Patent Document 6).

Dll4 has been reported to be expressed in differentiated myotubes (Non-Patent Document 7). Recently, Dll4 has been reported to be expressed also in other tissues such as microvascular endothelial cells (EC) of muscle tissue, and retina, etc. (Non-Patent Document 8, and Non-Patent Document 9).

### SUMMARY OF THE INVENTION

### Technical Problem

An object of the present invention is to provide a new therapeutic agent for muscular atrophy.

### Solution to Problem

The present inventors have found that Delta-Like Protein 4 (Dll4) activates Notch2 in myofibers and induces muscle atrophy, and that muscle atrophy can be suppressed as a result of suppressing Notch2 activation by Dll4, leading to completion of the present invention. The present invention includes the following.
[1] A preventive or therapeutic agent for muscular atrophy comprising a Dll4 function inhibitor (preferably a Dll4 function inhibitor secreted from endothelial cells, preferably a Dll4 function inhibitor secreted from vascular endothelial cells) as an active ingredient.
[2] The preventive or therapeutic agent for muscular atrophy according to claim 1, wherein the Dll4 inhibited by the Dll4 function inhibitor is Dll4 expressed in endothelial cells of blood vessels present in muscle tissue.
[3] The preventive or therapeutic agent for muscular atrophy according to claim 1 or 2, wherein the Dll4 function inhibitor is a binding inhibitor between Dll4 and Notch2.
[4] The preventive or therapeutic agent for muscular atrophy according to claim 3, wherein the binding inhibitor is an anti-Dll4 antibody that inhibits binding of Dll4 to Notch2.
[5] The preventive or therapeutic agent for muscular atrophy according to claim 4, wherein the anti-Dll4 antibody is an antibody that recognizes 5 to 10 amino acids selected from the amino acid sequence described in Dll4 amino acid sequence information (NP_061947.1).
[6] The preventive or therapeutic agent for muscular atrophy according to claim 1 or 2, wherein the Dll4 function inhibitor is a Dll4 expression inhibitor.
[7] The preventive or therapeutic agent for muscular atrophy according to claim 6, wherein the Dll4 expression inhibitor is siRNA or shRNA that induces RNA interference with Dll4 mRNA.
[8] The preventive or therapeutic agent for muscular atrophy according to claim 1 or 2, wherein the Dll4 function inhibitor is a collagen binding inhibitor to Dll4.
[9] The preventive or therapeutic agent for muscular atrophy according to any one of claims 1 to 8, wherein the muscular atrophy is neurogenic amyotrophic disease or myogenic amyotrophic disease.
[10] The preventive or therapeutic agent for muscular atrophy according to claim 9, wherein the muscular atrophy is selected from the group consisting of amyotrophic lateral sclerosis (ALS), myasthenia gravis, progressive muscular atrophy, spinal muscular atrophy, spinobulbar muscular atrophy, Guillain-Barre syndrome, and muscle atrophy due to physical spinal cord injury.
[11] The preventive or therapeutic agent for muscular atrophy according to claim 9, wherein the muscular atrophy is selected from the group consisting of age-related muscular atrophy (sarcopenia), disuse muscular atrophy, muscular dystrophy, diabetic muscular atrophy, congenital myopathy, distal myopathy, steroid myopathy, drug-induced myopathy, rhabdomyolysis, spinal muscular atrophy, progressive spinobulbar muscular atrophy, muscle wasting disease, Charcot-Marie-Tooth disease, Lambert-Eaton syndrome, muscle atrophy induced by cachexia (for example, cancer cachexia, myocardial infarction, renal failure, COPD, sepsis, HIV infection), and muscle atrophy associated with burns and the like.

### Advantageous Effect of the Invention

The present invention provides a preventive or therapeutic agent for muscular atrophy based on a new mechanism of action.

### BRIEF DESCRIPTION OF DRAWINGS

FIG. 1 is a diagram showing culture conditions for satellite cells isolated from mouse EDL muscle.

In FIG. 2, the left figure shows Notch2 gene expression level, the middle figure shows body weight, and the right figure shows muscle mass (n=3 (Cont), n=5 (N2-mTg)). Data represent mean±SEM, and points indicate individual mice (unless otherwise stated, the same applies to the following figures). * indicates P<0.05, ** indicates P<0.01, *** indicates P<0.001, and n.s. indicates no significant difference (unless otherwise specified, the same applies to the following drawings).

FIG. 3 shows the result of analyzing expression of the Notch2 gene in individual myofibers isolated from EDL muscle of Notch2^{flox/flox} (N2^{f/f}) and Mlc1f^{Cre/+}; Notch2^{-/-} (N2^{-/-}) mice by qPCR (n=5).

In FIG. 4, a shows an overview of a hindlimb unloading (HU) test. b is the result of analyzing Notch2 expression in myofibers isolated from Plantaris (PLA) (n=5). c is the result of measuring HU-induced muscle weight loss of soleus muscle (SOL), PLA, and gastrocnemius muscle (GAS) in N2^{f/f} and N2^{-/-} mice (n=5). Each left bar shows the result from N2^{f/f} mice, and each right bar shows the result from N2^{-/-} mice. d is a representative image of laminin immunostaining on tissue sections of PLA muscle after HU (scale bar=100 μm). e shows the myofiber cross-sectional area (CSA) of PLA muscle (n=5).

In FIG. 5, a shows an overview of a diabetes-induced muscle atrophy model test. b shows blood glucose levels 14 days after STZ injection (n=5-8). c is the result of measuring Notch2 expression in myofibers isolated from EDL (n=6). d is the result of measuring muscle weight loss in tibialis anterior (TA), PLA, and GAS 14 days after STZ injection (n=8). Each left bar shows the result from N2^{f/f} mice, and each right bar shows the result from N2^{-/-} mice. e is a representative image of laminin immunostaining of TA muscle cross section of diabetes-induced N2^{f/f} and N2^{-/-} mice (scale bar=100 μm). f shows the myofiber cross-sectional area (CSA) of TA myofibers (n=5).

FIG. 6 shows the result of measuring relative muscle weight loss in N2^{f/f} Akita mice or N2^{-/-} Akita mice (n=3). Each left bar shows the result from N2^{f/f} mice, and each right bar shows the result from N2^{-/-} mice.

FIG. 7 shows the result of measuring the muscle strength generated by tetanus stimulation in the TA muscles of N2^{f/f} Akita mice and N2^{-/-} Akita mice (n=4), under DB conditions. Representative tetanic force recordings (100 Hz) (left figure) and quantified maximal muscle strength (right figure) are shown.

FIG. 8 shows a representative transmission electron microscopy (TEM) image of longitudinal GAS muscle sections under DB conditions. Enlarged images boxed on the right represent T-tubules (upper figure) and sarcomere structures (lower figure), respectively. Arrows indicate abnormal mitochondria (scale bar=1 μm).

In FIG. 9, the upper figure shows the number of significantly upregulated genes common to both HU and DB conditions in GAS muscle of N2^{f/f} mice and N2^{-/-} mice (number of genes with fold change>2.0, q<0.05, and TPM>500). The lower figure shows the top 5 pathways from KEGG pathway analysis of common upregulated genes (361 genes) between HU and DB conditions in N2^{f/f} mice.

In FIG. 10, the left figure shows a representative image of myofibers stimulated with each protein (scale bar=50 μm). The right figure shows quantitative data of myofiber diameter (n=5 mice per condition, >15 myofibers were counted for each mouse).

FIG. 11 shows the result of gene expression analysis after 24 hours of culture on plates coated with Dll4 recombinant protein (n=5). The left bar of each shows the result of Cont and the right bar shows the result of rDll4.

In FIG. 12, the upper figure is a schematic figure of the co-culture conditions. The bottom left figure is a representative image after co-culture (scale bar=50 μm). The bottom right figure shows quantitative data of myofiber diameter (n=4 mice per condition, >15 myofibers were counted for each mouse).

In FIG. 13, the left figure shows the result of culturing on plates coated previously with recombinant Dll4 protein for EDL myofibers derived from N2^{f/f} mice and N2^{-/-} mice. The right figure shows the result of culturing with addition of DAPT on plates coated previously with recombinant Dll4 protein for EDL myofibers derived from N2^{f/f} mice. The upper figure is a representative image of a myofiber (scale bar=50 μm). The lower figure shows quantitative data of myofiber diameter (n=4 mice per condition, >15 myofibers were counted for each mouse). The left side of each shows the result of Cont and the right side shows the result of rDll4.

In FIG. 14, the left figure shows an outline of the test. The right figure shows the result of measuring the muscle mass of SOL, PLA, and GAS. The left bar of each shows the result of Cont, and the right bar shows the result of DAPT.

In FIG. 15, the left figure shows an outline of the test. The central figure shows the blood glucose concentration, and the right figure shows the result of measuring the muscle mass of SOL, PLA and GAS. The left bar of each shows the result of Cont, and the right bar shows the result of DAPT.

In FIG. 16, the left figure shows expression of Dll4 in CD31⁺ endothelial cells derived from freshly isolated myofibers and muscle tissue (n=4). The right figure shows expression of Notch ligands in CD31⁺ endothelial cells derived from wild-type GAS muscle in HU and DB conditions (n=4-6 mice for each condition).

FIG. 17 shows a representative image of CD31 and Dll4 immunohistochemical staining of TA muscle longitudinal sections derived from wild-type mice under HU and DB conditions (scale bar=20 μm).

FIG. 18 shows a representative confocal microscopy image (A) and a representative immunoelectron microscopy image (B) showing Dll4 protein distribution in EC in GAS muscle tissue of DB mice. Confocal microscopy images (scale bar=20 μm) visualize Dll4 and CD31, corresponding to Dll4⁺ CD31⁺ EC and Dll4⁻ CD31⁺ EC, respectively (white arrow (p) and arrowhead (q) correspond to immunoelectron microscopy images (scale bar=5 μm) of Dll4-positive CD31⁺ EC (figure B) and Dll4-negative CD31⁺ EC (figure C), respectively). Figure B is an image of DAB staining of Dll4 protein visualized by immunoelectron microscopy (right figure of figure B is an enlarged image), showing Dll4 protein (red arrow) accumulated on the myofiber surface. In enlarged images, Dll4 signals were detected in collagen-like fibers (green arrowheads). Figure C is a DAB-stained image of Dll4-negative EC (scale bar=5 μm) (negative control).

In FIG. 19, A is a conceptual figure of the transwell culture system used in this example. B shows the result of measuring Dll4 expression in EC transfected with siCont and siDll4. C is a representative image of myofibers (scale bar=50 μm). D shows quantitative data of myofiber diameter (n=4 mice per condition, >20 myofibers were counted for each mouse).

In FIG. 20, the left figure is a representative image of myofibers when EC was co-cultured with control antibody (Cont) and anti-Dll4 neutralizing antibody (scale bar=50 μm). The right figure shows quantitative data of myofiber diameter (n=4 mice per condition, >15 myofibers were counted for each mouse).

In FIG. 21, a shows an overview of a test in which a neutralizing antibody is administered to a wild-type mouse HU model. b shows the number of satellite cells per EDL myofiber on day 7 in antibody-treated mice (n=4). c shows a decrease in muscle mass of SOL, PLA, GAS muscles (n=7). The left bar indicates αCont and the right bar indicates αDll4, respectively.

In FIG. 22, d shows a representative image of laminin immunohistochemical staining of PLA muscle cross section. e shows quantitative data of myofiber CSA (n=6). f shows expression of FoxO target genes and muscle atrophy-causing genes in GAS muscle (n=6). In four bars, the leftmost bar represents αCont-GC, the second bar represents αCont-HU, the third bar represents αDll4-GC, and the rightmost bar represents αDll4-HU.

In FIG. 23, a shows an outline of a test in which a neutralizing antibody is administered to a wild-type mouse DB model. b shows blood glucose 14 days after STZ injection (n=5-7). c shows DB-induced muscle weight loss in TA, PLA, GAS muscles (n=5-6). The left bar shows αCont and the right bar shows αDll4, respectively.

In FIG. 24, d shows a representative image of laminin immunohistochemical staining of TA muscle cross section. e shows quantitative data of myofiber CSA (n=5). f shows expression of FoxO target genes and muscle atrophy-causing genes in GAS muscle (n=5). In the four bars, the leftmost bar indicates αCont-Veh, the second bar indicates αCont-DB, the third bar indicates αDll4-Veh, and the rightmost bar indicates αDll4-DB.

In FIG. 25, a shows an outline of a test of administering soluble Dll4-Fc recombinant protein to a wild-type mouse DB model. b shows blood glucose 14 days after STZ injection (n=5). c shows DB-induced decrease in muscle mass in TA, PLA, GAS (n=5). The left bar indicates PBS and the right bar indicates rDll4-Fc, respectively.

In FIG. 26, a shows an overview of a test in which a neutralizing antibody is administered to a wild-type mouse STZ-induced severe hyperglycemia model. b shows grip strength on day 7 (left) or day 14 (right) of STZ injection (n=9-10). c shows a survival curve (n=10). P-value for the survival experiment was determined using the Log-rank test.

In FIG. 27, a shows an overview of a test in which a neutralizing antibody is administered to a wild-type mouse mechanical load-induced muscle hypertrophy model. b shows a PLA muscle weight increase induced by overload (PLA muscle weight/body weight: OL-treated group (OL) vs untreated group (sedentary control: Sed)) (n=6). c shows a representative image of laminin immunohistochemical staining of overloaded PLA muscle cross section. d shows quantitative data of myofiber CSA (n=6).

In FIG. 28, a shows an outline of a test in which mechanical load (OL) is applied to N2^{f/f} and N2^{-/-} mice to induce muscle hypertrophy. Analysis was performed 14 days after tenotomy. b and c show overload-induced PLA muscle weight and weight gain (PLA muscle weight/body weight: OL-treated group (OL) vs untreated group (sedentary control: Sed)) (n=6-8).

In FIG. 29, a shows an overview of a test in which DAPT was administered in a wild-type mouse mechanical load (OL)-induced muscle hypertrophy model. DAPT (10 mg/kg) was administered intraperitoneally after tenotomy and analyzed on day 14. b and c show overload-induced PLA muscle weight and weight gain (PLA muscle weight/body weight: OL-treated group (OL) vs untreated group (sedentary control: Sed)) (n=6).

FIG. 30 is a representative TEM image of immunogold labeling of Dll4 in GAS muscle isolated from diabetic mice. Gold particles were localized near myofibers (a), collagen-like fibers (b), or extracellular vesicles (c) in the interstitial space between EC and myofibers. Scale bar=200 nm.

### DESCRIPTION OF EMBODIMENTS

Hereinafter, the present invention will be illustrated in more detail along with preferred methods and materials which can be used in practice of the present invention. However the present invention is not limited to the following embodiments. Unless otherwise specified in the sentences, any technical terms and scientific terms used in the present specification have the same meaning as those generally understood by those of ordinary skill in the art to which the present invention belongs. Any materials and methods equivalent or similar to those described in the present specification can be used for practicing the present invention. All publications and patents cited herein in connection with the present invention described herein are incorporated by reference, for example, as indicating methodology, materials, etc. that can be used in the present invention.

In the present specification, the description of “A to B” indicating a numerical range means a numerical range including A and B as endpoints. The same applies to “A through B”. Moreover, in the present specification, the term “about” is used in the sense of allowing ±10%.

In the present specification, the preventive or therapeutic agent for muscular atrophy containing a Dll4 function inhibitor as an active ingredient of the present invention is sometimes referred to as the “preventive or therapeutic agent of the present invention”, the “pharmaceutical composition of the present invention”, or the “composition of the present invention”. In the present specification, the active ingredient that can be used in the preventive or therapeutic agent of the present invention is sometimes referred to as the “active ingredient of the preventive or therapeutic agent of the present invention”, the “active ingredient of the pharmaceutical composition of the present invention”, the “active ingredient of the composition of the present invention”, or the “active ingredient of the present invention”, and when apparent from the context, may simply be referred to as the “active ingredient”. In the present specification, the description “Dll4 function inhibitor” is used in the sense of including any of an inhibitor for binding of Dll4 to Notch2, a Dll4 expression inhibitor, and an inhibitor for binding of collagen against Dll4, unless it is clear from the context which one is indicated. In the present specification, Dll4 and Dll4 protein refer to Dll4 as a substance (protein) and are used as interchangeable terms, unless the context clearly indicates another meaning. In the present specification, Notch2 and Notch2 protein refer to Notch2 as a substance (protein) and are used as interchangeable terms, unless the context clearly indicates another meaning.

The present invention is a preventive or therapeutic agent for muscular atrophy containing a Dll4 function inhibitor as an active ingredient. In the present invention, “Dll4 function inhibitor” means a substance capable of suppressing muscle atrophy by inhibiting the function of Dll4 that causes muscle atrophy based on the Dll4-Notch2 signaling axis. The present inventors have found that microvascular endothelium in muscle tissue upregulates and secretes Dll4 which is a ligand for Notch2, Dll4 secreted from vascular endothelial cells binds to Notch2 present on the surface of myofibers without direct cell-to-cell contact, thereby, Notch2 is selectively activated, resulting in induction of muscle atrophy signals, to cause muscle atrophy. The present inventors have also found that it is important that Dll4 secreted from endothelial cells, which is a ligand, binds to collagen in order to bind to Notch2 as a receptor. The present inventors have further found that Dll4 is expressed in the membrane of extracellular vesicles secreted by endothelial cells, and muscle atrophy caused by activation of Notch2 by Dll4 occurs via the extracellular vesicles. The present inventors have also found that Dll4 secreted from endothelial cells has a cleaved type with a smaller molecular weight than originally assumed Dll4, and that this cleaved type Dll4 is important for binding to Notch2 and activation. Therefore, in the present invention, “Dll4 function inhibitor” means a substance that inhibits the action of Dll4 that induces muscle atrophy via Notch2, and is used in the sense of including any of, but not limited to, for example, a substance that inhibits binding between Dll4 and Notch2 (preferably a substance that inhibits binding between Dll4 secreted from blood vessels and Notch2 in myofibers), a substance that inhibits Dll4 expression (including, e.g., a Dll4 expression inhibitor in endothelial cells, a Dll expression inhibitor in extracellular vesicles), and a substance that inhibits the binding of Dll4 and Notch2 by inhibiting the binding of collagen to Dll4.

A substance that inhibits the binding between Dll4 and Notch2 (hereafter, referred to as “binding inhibiting substance between Dll4 and Notch2”, “binding inhibitor between Dll4 and Notch2”, or if apparent from the context, referred to as simply “binding inhibiting substance” or “binding inhibitor”) inhibits the binding between Dll4 and Notch2, thereby suppressing activation of Notch2, and suppresses the induction of muscle atrophy signals, to suppress muscle atrophy. As a result, it can be used as an active ingredient of a preventive or therapeutic agent for muscle atrophy. Binding inhibiting substances include, for example, but are not limited to, substances that bind to Dll4 and inhibit physical binding of Dll4 to Notch2. Specifically, small molecules, middle molecules, antibodies and the like that bind to a site of Dll4 binding to Notch2 (hereafter, sometimes referred to as “Dll4 to Notch2 binding site”, “Notch2 binding site”, or “receptor binding site”) can be mentioned. Antibodies that recognize the binding site of Dll4 to Notch2 are preferred. Binding inhibiting substances include, for example, but are not limited to, substances that bind to Notch2 and inhibit physical binding of Notch2 to Dll4. Specifically, small molecules, middle molecules, antibodies and the like that bind to a site of Notch2 binding to Dll4 (hereinafter sometimes referred to as “Notch2 to Dll4 binding site”, “Dll4 binding site”, or “ligand binding site”) can be mentioned. Antibodies that recognize the binding site of Notch2 to Dll4 are preferred.

Dll4 secreted from blood vessels binds to Notch2 in myofibers. Therefore, the binding inhibiting substance includes preferably substances that inhibit the binding between Dll4 secreted from blood vessels and Notch2 in myofibers. Examples of the substances that inhibit the binding between Dll4 and Notch2 also include binding inhibitors that target modification of sugar chain of the extracellular domain of Notch2, modification of the protein of Dll4, and the like.

Small molecule or middle molecule compounds can be searched for by screening using Dll4-binding activity, Notch2-binding activity, or activity that inhibits the binding between Dll4 and Notch2 as an index according to conventional methods. For example, by binding Dll4 and Notch2 in the presence or absence of a test substance, then, detecting the Dll4-Notch2 conjugate by ELISA using an anti-Dll4 antibody and an anti-Notch2 antibody, substances that inhibit the binding of Dll4 and Notch2 can be screened, but the method is not limited to this. The anti-Dll4 antibody and anti-Notch2 antibody can be prepared according to a conventional method, but commercially available ones may also be used.

Alternatively, a system can be used in which Dll4 chemically modified to emit fluorescence when Dll4 molecules bind to Notch2 molecules is used, isolated myofibers are adherently cultured, and binding of Dll4 to Notch2 expressed in myofibers is detected by fluorescence. Alternatively, a system that detects binding of Dll4 and Notch2 molecules using a luciferase reporter assay can also be used. Furthermore, instead of isolated myofibers, cells in which Notch2 is excessively expressed on the cell surface can be used in the same manner.

An antibody that recognizes the binding site of Dll4 to Notch2 or an antibody that recognizes the binding site of Notch2 to Dll4 can be prepared by a conventional method using Dll4 protein or Notch2 protein. Alternatively, it can be prepared by a conventional method using genetic engineering techniques with reference to Dll4 amino acid sequence information or Notch2 amino acid sequence information. Nucleic acid and amino acid sequences of Dll4 and Nothc2 are available from known databases, respectively. Human Dll4 has been registered as nucleic acid sequence (NM 019074.4) and amino acid sequence (NP_061947.1), and human Notch2 has been registered as nucleic acid sequence (NM 024408.4) and amino acid sequence (NP_077719.2). For example, reports on Dll4 and Notch2 binding sites (e.g., Non-Patent Document 10, Non-Patent Document 11) can be referred to generate antibodies against amino acid sequences that are believed to contain the binding sites. In addition, for example, anti-Notch2 antibodies include, but not limited to, anti-Notch2 antibodies that bind to the region of EGF repeats 11-13 of Notch2, since it has been suggested that Dll4 binds to the region of EGF repeats 11-13 of the extracellular domain of Notch2. Antibodies may be full-length (e.g., IgG1, IgG2 or IgG4), or include their antigen-binding sites and may be modified to improve functionality or remove nonessential effector functions. Examples thereof include other engineered molecules such as disulfide Fvs, dAb fragments, fragments containing complementarity determining regions (CDRs), single domains such as isolated CDR fragments, restricted FR3-CDR3-FR4 peptides, single chain antibodies (scFv), mini bodies, nanobodies (e.g., monovalent nanobodies, bivalent nanobodies, etc.), diabodies, domain deleted antibodies, chimeric antibodies, CDR-grafted antibodies, bispecific antibodies, multispecific antibodies (e.g., trispecific antibodies, tetraspecific antibodies), polypeptides comprising at least a portion of an antibody with specific antigen-binding ability, and small modular immunopharmaceuticals. Alternatively, it may be an antigen-binding fragment of an antibody. The antigen-binding fragment of an antibody includes polypeptided or glycoproteins that form a complex by specifically binding to Notch2 binding site of Dll4 which is any naturally occurring or synthetized, obtained using enzymatic or biochemical means, or obtained by genetic engineering. The above content is the same when Notch2 is used as an antigen.

The antigen-binding fragment of an antibody can be obtained from a complete antibody molecule or its amino acid or genetic information using any suitable standard technique, such as, for example, protein digestion, or recombinant genetic engineering techniques involving the manipulation and expression of the DNA encoding the variable and (optionally) constant domains of the antibody. The DNA is sequenced and chemical or molecular biological techniques are used to place, for example, one or more variable and/or constant domains into proper positions, alternatively, for example, arbitrary codons are introduced to create cysteine residues or amino acids are modified, added or deleted, thus, any antigen-binding fragments can be generated by the above operations and the like.

The antigen-binding fragment of an antibody typically comprises at least one variable domain. The variable domain can be of any size or amino acid composition and generally comprises at least one CDR, which is flanked by or in-frame with one or more framework sequences. In an antigen-binding fragment having a VH domain joined to a VL domain, the VH and VL domains are relatively positioned in any suitable arrangement. The antigen-binding fragment of an antibody includes, for example, variable regions that are dimeric, VH-VH, VH-VL or VL-VL dimers. Alternatively, the antigen-binding fragment of an antibody comprises monomeric VH or VL domains.

In certain embodiments, the antigen-binding fragment of an antibody contains at least one variable domain covalently bind to at least one constant domain. Typical arrangements of variable and constant domains within antigen-binding fragments of antibodies include, but are not limited to: (i) VH-CH1; (ii) VH-CH2; (iii) VH-CH3; (iv) VH-CH1-CH2; (v) VH-CH1-CH2-CH3; (vi) VH-CH2-CH3; (vii) VH-CL; (viii) VL-CH1; (ix) VL-CH2; (x) VL-CH3; (xi) VL-CH1-CH2; (xii) VL-CH1-CH2-CH3; (xiii) VL-CH2-CH3; and (xiv) VL-CL. In any arrangement of variable and constant domains, including any of the exemplary arrangements described above, the variable and constant domains are either directly bonded to each other or linked by a linker region. Linker regions consist of at least 2 (e.g., 5, 10, 15, 20, 40, 60 or more) amino acids and link adjacent variable and/or constant domains. Additionally, the antigen-binding fragment of an antibody includes homo-dimers or hetero-dimers (or other multimers) of any of the variable and constant domain arrangements described above, in which one or more monomeric VH or VL domains are non-covalently associated.

As with whole antibody molecules, antigen-binding fragments can be monospecific or multispecific (e.g., bispecific). The multispecific antigen-binding fragment of an antibody typically comprises at least two different variable domains, each variable domain being capable of specifically binding to different antigens or different epitopes on the same antigen. Any multispecific antibody, including bispecific antibodies herein, can be made using conventional techniques available in the art, and can be used as the antigen-binding fragment of an antibody for the purposes of the present invention.

A substance that inhibits expression of Dll4 (hereinafter, referred to as “Dll4 expression inhibiting substance”, “Dll4 expression inhibitor”, or if apparent from the context, referred to as simply “expression inhibiting substance” or “expression inhibitor”) inhibits Dll4 expression, thereby reducing expression of Dll4, for example, expression of Dll4 in endothelial cells and expression of Dll4 in extracellular vesicles, thereby suppressing activation of Notch2 by Dll4, and suppresses the induction of muscle atrophy signals, to suppress muscle atrophy. Preferably, Dll4 expression in endothelial cells, more preferably Dll4 expression in vascular endothelial cells is decreased. As a result, it can be used as an active ingredient of a preventive or therapeutic agent for muscle atrophy. Expression inhibiting substances include, for example, nucleic acid molecules that specifically inhibit expression of Dll4. Nucleic acid molecules that specifically inhibit expression of Dll4 are typically non-naturally occurring nucleic acids, such as RNAi, antisense nucleic acids, or ribozymes that specifically block expression of Dll4. By “RNAi” is meant any RNA capable of down-regulating the expression of Dll4 as a target subject, and siRNA, dsRNA, SSRNA and shRNA molecules are included. Preferably, the Dll4 expression inhibitor is siRNA or shRNA.

siRNAs or shRNAs are usually designed against a region 19-50 nucleotides downstream of the translation initiation codon. In a preferred embodiment, the RNAi molecule is an siRNA consisting of at least about 10-40 nucleotides, preferably about 15-30 nucleotides in length. siRNAs or shRNAs can include naturally occurring RNAs, synthetic RNAs, or recombinantly produced RNAs, as well as modified RNAs that differ from naturally occurring RNAs by the addition, deletion, substitution, and/or alteration of one or more nucleotides. Such modifications include modifications that render the siRNA resistant to nuclease digestion, and can include, for example, addition of non-nucleotide substances to the ends of the molecule or to one or more internal nucleotides of the siRNA, etc.

Nucleic acids that are expression inhibitors of the present invention have the ability to specifically hybridize to Dll4-encoding genes or transcripts. Nucleic acids as expression inhibitors do not require 100% complementarity with Dll4 sequence as the target sequence, but preferably have at least equal to about 90% complementarity to specifically hybridize. More preferably, the complementarity between the nucleic acid as the expression inhibiting substance in the present invention and the target sequence (Dll4 target sequence) is at least 958, 96%, 97%, 98%, 99%, or 100%.

A substance that inhibits binding of collagen to Dll4 (hereinafter referred to as “Dll4 to collagen binding inhibiting substance”, “Dll4 to collagen binding inhibitor”, or if apparent from the context, referred to as simply “binding inhibiting substance” or “binding inhibitor”) inhibits the binding of Dll4 secreted from vascular endothelial cells to collagen, thereby inhibiting the binding of Dll4 to Notch2 to suppress activation of Notch2, and suppresses the induction of muscle atrophy signals, to suppress muscle atrophy. As a result, it can be used as an active ingredient of a preventive or therapeutic agent for muscle atrophy. Binding inhibiting substances include, for example, but are not limited to, substances that bind to Dll4 and inhibit physical binding of Dll4 to collagen. Specifically, small molecules, middle molecules, antibodies, etc., that bind to a site of Dll4 binding to collagen (hereinafter sometimes referred to as “Dll4 to collagen binding site” or “collagen binding site”) can be mentioned. Preferably, an antibody that recognizes the binding site of Dll4 to collagen can be mentioned. Binding inhibiting substances include, for example, but are not limited to, substances that bind to collagen and inhibit the physical binding of collagen to Dll4. When using an antibody, various antibodies can be used, as described in antibodies that inhibit the binding of Dll4 and Notch2.

Examples of other Dll4 function inhibitors include substances that inhibit cleavage of the intracellular domain of Notch2 (activation of Notch2) even when Dll4 binds to the extracellular domain of Notch2. Such inhibiting substances include, but are not limited to, substances that inhibit metalloproteases and secretases associated with Notch2 cleavage.

The composition containing a Dll4 function inhibitor as an active ingredient of the present invention is used for the prevention or therapy of diseases associated with muscle atrophy. The composition of the present invention is further used to improve muscle atrophy, thereby improving muscle mass loss or muscle weakness. For example, maintenance of muscles weakened by aging and improvement of urinary incontinence, and the like, can be mentioned. Muscle atrophy refers to the loss of muscle mass, resulting in a decrease in muscle mass. The “muscular atrophy” targeted by the preventive or therapeutic agent of the present invention includes both neurogenic amyotrophic diseases and myogenic amyotrophic diseases.

The neurogenic amyotrophic diseases include, for example, amyotrophic lateral sclerosis (ALS), myasthenia gravis, progressive muscular atrophy, spinal muscular atrophy, spinobulbar muscular atrophy, Guillain-Barre syndrome, muscle atrophy due to physical spinal cord injury, and the like.

The myogenic amyotrophic diseases include, for example, age-related muscular atrophy (sarcopenia), disuse muscular atrophy, muscular dystrophy (e.g., Duchenne muscular dystrophy, Becker muscular dystrophy, distal muscular dystrophy, congenital muscular dystrophy, Emery-Dreifuss muscular dystrophy, facioscapulohumeral muscular dystrophy, myotonic muscular dystrophy type 1 or myotonic muscular dystrophy type 2, limb girdle muscular dystrophy), diabetic muscular atrophy, congenital myopathy, distal myopathy, steroid myopathy, drug-induced myopathy, rhabdomyolysis, spinal muscular atrophy, progressive spinobulbar muscular atrophy, muscle wasting disease (e.g., age-related sarcopenia, cachexia), Charcot-Marie-Tooth disease, Lambert-Eaton syndrome, muscle atrophy induced by cachexia (e.g., cancer cachexia, myocardial infarction, renal failure, COPD, sepsis, HIV infection), muscle atrophy associated with burns etc.; and the like.

The preventive or therapeutic agent for muscular atrophy of the present invention contains a therapeutically effective amount of a Dll4 function inhibitor. The “therapeutically effective amount” refers to an amount sufficient to produce a therapeutic effect when administered to a mammal in need of treatment. The therapeutically effective amount will vary depending on the subject and the disease symptom to be treated, the subject's weight and age, the severity of the disease symptom, the mode of administration, etc., and can be readily determined by those skilled in the art. In certain embodiments, Dll4 function inhibitors are small or medium molecules. In certain embodiments, the Dll4 function inhibitor is an antibody or antigen-binding fragment thereof, which is used in substantially purified form. In certain embodiments, the Dll4 function inhibitor is a nucleic acid, preferably siRNA or shRNA. In certain embodiments, Dll4 function inhibitors of the present invention are administered preventively to a subject suffering from muscle atrophy. In certain embodiments, Dll4 function inhibitors of the present invention are administered therapeutic purposes to a subject suffering from muscle atrophy.

Subjects to whom the preventive or therapeutic agent of the present invention is administered are mammals, preferably humans. In a more specific embodiment, the subject is a human suffering from muscular atrophy. In addition to therapeutic purposes, the compositions of the present invention can be administered to subjects suffering from mild or moderate muscular atrophy preventively to prevent the progression of muscular atrophy. The compositions of the present invention may be administered primarily for therapeutic purposes to subjects suffering from moderate or severe muscular atrophy.

Compositions of the present invention comprise a therapeutically effective amount of a DDL4 function inhibitor and further comprise a pharmaceutically acceptable carrier. In the present specification, “pharmaceutically acceptable carrier” means a carrier approved for use by a regulatory agency or listed in a generally accepted pharmacopoeia for use in animals, particularly humans, and includes any and all solvents, dispersion media, coating agents, antioxidants, chelating agents, preservatives (e.g., antibacterial agents, antifungal agents), surfactants, buffers, osmo-regulator, absorption delaying agents, salts, drug stabilizers, excipients, diluents, binders, disintegrants, sweeteners, flavoring agents, lubricants, dyes, etc., and combinations thereof, as known to those skilled in the art. Except insofar as any carrier is incompatible with the active ingredient of the invention, it can be used in the compositions of the invention.

The composition of the present invention can be appropriately formulated into a desired dosage form according to an administration method such as oral administration or parenteral administration. The dosage form is not particularly limited, but in the case of oral administration, for example, solid preparations such as powders, granules, tablets, troches, capsules, and the like; liquid preparations such as solutions, syrups, suspensions, emulsions, and the like, can be formulated. For parenteral administration, it can be formulated into, for example, suppositories, sprays, inhalants, ointments, patches, injections (including infusions), and the like. The composition of the present invention may be a freeze-dried preparation that is dissolved by adding sterilized water or the like at the time of use. In addition, formulation can be suitably implemented by a known method according to a dosage form.

In one embodiment, the present invention is a method for preventing or treating a subject by administering to the subject a therapeutically effective amount of a Dll4 function inhibitor. The therapeutically effective amount, administration method, and other aspects of the DDL4 function inhibitor are as described above for the preventive and therapeutic agents.

The dose of the DDL4 function inhibitor, which is the active ingredient of the present invention, is appropriately selected according to the type of the DDL4 function inhibitor. In addition, the dosage of the DDL4 function inhibitor, which is the active ingredient of the present invention, is appropriately selected depending on the symptoms, age, condition, route of administration, and the like of the subject to be administered.

When a small molecule or middle molecule is used as a DDL4 function inhibitor, which is an active ingredient of the present invention, the composition of the present invention can be administered orally or parenterally as a medicament, preferably administered orally. For oral administration, solid formulations such as tablets, powders, capsules, granules, and the like can be used, and these may contain conventional additives such as excipients, binders, diluents, lubricants, and the like. They can also be administered as solvents such as aqueous or oily suspensions, solutions, syrups, elixirs, or the like. In the case of parenteral administration, it can be used as an injection or the like. The dosage varies depending on the patient's age, body weight and condition, type and degree of disease, administration methods, or the like, and cannot be categorically defined, but is usually about 0.1 to about 500 mg/kg per day for an adult.

When an antibody is used as a DDL4 function inhibitor, which is the active ingredient of the present invention, for the prevention or therapy of diseases in adult patients, usually, the lower limit is about 0.01 mg, preferably about 0.05 mg, and more preferably about 0.1 mg, while the upper limit is about 100 mg, preferably about 50 mg, more preferably about 20 mg, and further preferably about 10 mg, per kg body weight, administered in a single dose, although not limited thereto. When using the antigen-binding fragment of an antibody of the present invention, it is usual to administer a dose higher than the dose, for example, but not limited to, about 1.5-fold to about 50-fold, preferably about 1.5-fold to about 20-fold, and more preferably about 1.5-fold to about 10-fold the amount is administered. The frequency and interval of administration is adjusted depending on the severity of the condition. In certain embodiments, antibodies, etc., are administered in a starting dose of at least about 1 mg to about 1000 mg. In certain embodiments, the starting dose is followed by a second or multiple subsequent doses of an antibody or antigen-binding fragment thereof in an amount that is about the same as or less than that of the starting dose, in which the subsequent dose is separated by at least 2 to 5 days; at least 1 week; at least 2 weeks; at least 3 weeks; at least 4 weeks; at least 5 weeks; at least 6 weeks; at least 7 weeks; at least 8 weeks; at least 9 weeks; at least 10 weeks; or at least 12 weeks.

The compositions of the present invention, whether oral or parenteral, are prepared in dosage forms prepared in unit doses to suit the desired dosage of the active ingredient. Dosage forms containing unit doses include, for example, tablets, pills, capsules, injections (ampoules), suppositories, and the like. The amount of an antibody to be included is generally preferably from about 5 to about 1000 mg of the antibody or the like per dosage form in a unit dose.

Administration and treatment plans, including dosage and administration schedule, can be appropriately determined by the physician who diagnosed the subject (patient), taking into consideration the symptoms, age, condition, route of administration, etc. of the subject to be administered, if necessary, with reference to guidelines provided by various institutions.

The administration route of the pharmaceutical composition of the present invention can be arbitrarily determined in consideration of the symptom, state and other conditions of the subject. Routes of administration include, but are not limited to, intradermal, transdermal, intramuscular, intraperitoneal, intravenous, subcutaneous, intranasal, epidural and oral routes. The pharmaceutical compositions may be administered by any convenient route, such as by infusion or bolus injection, or by absorption through the epithelium or mucocutaneous lining (e.g., oral mucosa, rectal, intestinal mucosa, etc.), and may be administered together with other biologically active agents. Administration can be systemic or local.

Various delivery systems are known for administration of active pharmaceutical ingredients, and these can be used to administer the pharmaceutical composition of the present invention. Examples thereof include encapsulation in liposomes, microparticles, nanoparticles, and microcapsules. In certain embodiments, the pharmaceutical compositions can be delivered in a controlled release system. Controlled-release systems place the composition near the target, thus requiring only a small systemic dose.

When administering the pharmaceutical composition of the present invention by injection, injectable preparations include intravenous, subcutaneous, intradermal, intraperitoneal and intramuscular injection forms, or infusion forms and the like. These injectable preparations can be prepared with reference to known methods. Injectable preparations can be prepared, for example, by dissolving, suspending, or emulsifying the antibody of the present invention, etc., or a salt thereof in a sterile aqueous or oily medium commonly used for injection. Aqueous medium for injection include, for example, physiological saline, isotonic solutions containing glucose, and other adjuvants, and can also be used in combination with a suitable solubilizer such as alcohols (e.g., ethanol), polyhydric alcohols (e.g., propylene glycol, polyethylene glycol), nonionic surfactants [e.g., polysorbate 80, HCO-50 (polyoxyethylene (50 mol) adduct of hydrogenated castor oil)], etc. As an oily medium, for example, sesame oil, soybean oil and the like are used, and they can be used in combination with a solubilizer such as benzyl benzoate, benzyl alcohol and the like. Injections prepared are also preferably filled into suitable ampoules.

When the pharmaceutical composition of the present invention is an injection, it can be prepared as a ready-to-use injection or as a prefilled syringe. The pharmaceutical compositions of this invention are administered subcutaneously or intravenously with standard needles and syringes. A pen delivery device can also be used for subcutaneous administration. Pen delivery devices are either reusable or disposable.

When a nucleic acid, for example, siRNA is used as a DDL4 function inhibitor in the composition of the present invention, it may be a single siRNA or a mixture of multiple siRNAs (so-called cocktail).

When using the siRNA of the present invention in vivo, the siRNA can be directly injected into the affected area, or a vector capable of expressing the siRNA can be used. When injecting siRNAs directly into the affected area, they can be injected complexed with liposomes such as Lipofectamine, Lipofectin, Cellfectin and other positively charged liposomes.

When a vector capable of expressing siRNA is used, for example, an expression vector containing a DNA sequence encoding RNA containing a sense strand sequence of siRNA and its complementary antisense strand sequence under the control of a promoter is preferably used.

To obtain siRNA, shRNA can also be used or expressed intracellularly. The shRNA that can be used in the present invention comprises the sense strand sequence, the antisense strand sequence, and a single-stranded loop sequence that covalently binds between the sense strand sequence and the antisense strand sequence, and is an RNA that is processed by Dicer which is an intracellular RNase to form siRNA.

The shRNA may be a single shRNA or a mixture of multiple shRNAs (so-called cocktail).

Viral vectors may be used to deliver the siRNA to the affected area. Viral vectors that can be used are not particularly limited, and examples thereof include adenoviral vectors, adeno-associated viral vectors, lentiviral vectors, retroviral vectors (such as leukemia viral vectors), herpes viral vectors, and the like. Viral vectors are preferably of the type lacking self-replication ability, for example, so as not to cause disease when used in humans. For example, in the case of adenoviral vectors, self-replication-deficient adenoviral vectors lacking E1 and E3 genes can be used. Viral vectors can be constructed according to known methods.

Since the purpose of the present invention is to suppress the expression of the DDL4 gene in skeletal muscle, an adeno-associated viral vector having a selectivity for delivery to skeletal muscle is particularly preferably used. As adeno-associated viral vectors, types I to XI have been known so far, and these can be used without restriction, but vectors developed in the future can also be used without restriction as long as the object of the present invention is achieved. Preferred vectors are, but are not limited to, AAV1, AAV2, AAV4, AAV6, AAV7, AAV8, or AAV9. Various adeno-associated viral vectors that are suitably used are commercially available.

When a viral vector is used, the gene can be introduced into cells by directly injecting the vector into the affected area and infecting the cells. In particular, adeno viral vectors have been known to be capable of introducing genes into various cell types with very high efficiency, and are actually clinically applied for gene therapy. Also, since this vector does not integrate into the genome, its effects are considered to be transient and its safety is higher than that of other viral vectors.

The dosage for direct administration of siRNA or shRNA is not particularly limited, and an amount that produces the effect of causing a decrease in DDL4 at the protein level is selected. The dose is a therapeutically effective amount and can be administered multiple times at regular time intervals. When actually used, the dosage is determined by the judgment of a medical specialist according to the patient's condition, age, sex, severity, and the like.

The siRNA or shRNA-carrying vector is administered to the patient along with a pharmaceutically acceptable carrier such as physiological saline, buffers, and the like. Pharmaceutical compositions may further contain stabilizers, preservatives, tonicity agents, and the like. The administration method of the pharmaceutical composition of the present invention is not particularly limited, and can be either local or non-local administration, preferably local administration. For local administration, the vector can be administered directly by means of a syringe or the like. Non-local administration can be carried out, for example, by intravenous administration. As an administration form, for example, a viral vector or a plasmid-liposome complex is administered in a form suspended in a pharmaceutically acceptable carrier.

### EXAMPLES

The present invention will be specifically described below with reference to examples, but the present invention is not limited to the following examples.

The methods used in the examples of the present invention are described below.

### (1) Antibodies and Reagents

Various antibodies and reagents used in the examples were purchased from commercial sources.

### (2) Animals

All animal experimental procedures were approved by the Institutional Animal Care and Usage Committee (IACUC) of Kumamoto University. The animals were housed in a pathogen-free, temperature- and humidity-controlled environment. They were housed under a 12 hour light cycle with free access to food and water. Mice used in the Examples were commercially available mice, and if necessary, mice with specific gene phenotypes were bred to produce mice with desired gene phenotypes.

### (3) In Vitro Inhibition Test of Notch Signaling

Wild-type mice were used to examine the effects of inhibition of the Notch signaling pathway on muscle in vitro. Ultra-LEAF™ anti-mouse Dll4 antibody (Dll4 blocking antibody) or Ultra-LEAF™ American hamster IgG control antibody (control antibody) was administered intraperitoneally (10 mg/kg body weight) every 4 days in each experiment. Recombinant Dll4-Fc chimeric protein (rDll4) was dissolved in phosphate-buffered saline (PBS) and administered (0.5 mg/kg body weight) every two days. The γ-secretase inhibitor, DAPT, dissolved in 10% ethanol and corn oil, was administered every 2 days (10 mg/kg body weight). Control mice were injected intraperitoneally with an equivalent volume of vehicle in rDll4 or DAPT experiments.

### (4) Hindlimb Unloading Model by Tail Suspension

Mice were individually suspended from the tail in each cage by a cord tied at one end to the tail and the other to a custom cage lid. The length of the cord was adjusted so that the mice could freely move their forelimbs and take food and drink. Littermates and age-matched ground (non-suspended) mice were used as controls. After 7 days of suspension, mice were killed by cervical dislocation and the soleus (SOL), PLA, and gastrocnemius (GAS) muscles were harvested.

### (5) STZ-Induced Diabetes Model

Mice were given a single intraperitoneal injection of STZ (150 mg/kg body weight) at a level that is toxic to insulin-producing pancreatic β-cells, or an equal volume of solvent (0.1 M citrate buffer, pH 4.5) as a control was injected. Mice were monitored for hyperglycemia on day 3 using Accu-chek (Roche), and only mice that achieved casual blood glucose levels>300 mg/dl were used in the experiment. Mice were sacrificed 14 days after STZ injection.

For survival experiments, wild-type mice were given a single intraperitoneal injection of a high dose of STZ (250 mg/kg), which induces severe diabetes. These mice were then administered every 4 days with either a Dll4 neutralizing antibody or a control antibody (10 mg/kg body weight). The diabetic mice were monitored for their appearance daily and their body weight and blood glucose levels were measured weekly. Experiments were stopped when all mice in either group died or were euthanized.

### (6) Tenotomy

Tenotomy surgery was performed on both legs of mice to induce muscle hypertrophy. In anesthetized mice, an incision was made along the midline of the calf after hair removal. After excision of the tendons belonging to SOL and GAS, the skin was closed using 3 or 4 sutures. 14 days after overload, mice were sacrificed and PLA muscles were harvested.

### (7) Measurement of Grip Strength and Tetanus Muscle Strength

Whole limb grip strength was measured using a rat grip dynamometer. Peak tension (N) was recorded when the mouse released the grip strength. The maximum intensity determined from 10 consecutive measurements was used for data analysis.

Tetanus muscle strength was measured in the tibialis anterior (TA) using a whole animal muscle strength testing system. Briefly, mice were anesthetized with isoflurane and placed on a 37° C. heating plate throughout the procedure. The right leg was fixed to a footplate connected to a servomotor, and the right knee was fixed with a knee clamp. The TA muscle was stimulated subcutaneously with two needle electrodes at 5 mA. Tetanic contractions were induced by stimulation with a frequency of 100 Hz for 350 ms. Each contraction was followed by an interval of at least 2 minutes, and maximal muscle strength determined from three consecutive measurements was used for data analysis.

### (8) Isolation of Myofibers and Satellite Cell Culture

Myofibers were isolated from the extensor digitorum longus (EDL) or PLA, by enzymatic degradation using type I collagenase, with some modifications to the present inventor's previous report (Ono et al., Cell Report 10, 1135-1148 (2015)). For gene expression analysis, isolated myofibers were immediately frozen using liquid nitrogen. Satellite cells were obtained from myofibers isolated from EDL muscle. Satellite cells were culture in GM (DMEM supplemented with 30% fetal bovine serum (FBS), 1% chicken embryo extract, 10 ng/ml basic FGF, 1% penicillin-streptomycin) at 37° C. in an atmosphere containing 5% CO₂. Myogenic differentiation was induced by replacing the medium with DM (DMEM supplemented with 2% horse serum and 1% penicillin-streptomycin). 40H TMX (1 μM) was used to induce Cre recombinase expression in differentiated myotubes.

### (9) EC Isolation Culture

Primary endothelial cells (EC) were isolated from muscle tissue of wild-type mice using Dynabeads, with slight modification to Muramatsu et al.'s report (Arteriosclerosis, thrombosis, and vascular biology 40, 2425-2439 (2020)). Anesthetized mice were perfused with PBS and hindlimb muscle tissue was harvested. Muscle tissue was digested with 0.2% type II collagenase followed by red blood cell lysis. Cells were incubated with Dynabeads conjugated with anti-CD31 antibody and separated into endothelial and non-endothelial fractions using a magnetic separation system. After the first selection, cells were cultured for 7 days in DMEM (supplemented with 20% FBS, nonessential amino acids, sodium pyruvate, 25 mM HEPES, 0.1 mg/ml heparin, 0.1 mg/ml endothelial cell growth supplement, and 1% penicillin-streptomycin). For the second selection, expanded cells were harvested and incubated with Dynabeads conjugated with anti-CD102 antibody to increase EC purity. Purified primary ECs were cultured to confluence for co-culture with myofibers. To reduce expression of Dll4, EC was transfected with 20 nM Dll4-siRNA (SASI_Mm01_00092621, MilliporeSigma) or control-siRNA (MilliporeSigma) using Lipofectamine RNAiMAX.

### (10) Plasmid Transfection

The mouse Dll1 cDNA and mouse Dll4 cDNA were cloned into the pcDNA3.1+P2A-eGFP plasmid vector (GenScript), to generate pcDNA3.1+Dll1-P2A-eGFP (Dll1-GFP) and pcDNA3.1+Dll4-P2A-eGFP (Dll4-GFP). HEK293 cells were transfected with either Dll1-GFP plasmid, Dll4-GFP plasmid, or pcDNA3.1+P2A-eGFP control plasmid, and Lipofectamine LTX was used with Plus reagent according to the manufacturer's instructions. GFP signal was captured using a fluorescence microscope.

### (11) Myofiber Culture and Diameter Measurement

Culture dishes were coated with Notch ligand recombinant protein as reported by Sakai et al. (Sakai et al., PLOS One 12, e0177516 (2017)). Culture dishes were first coated with 10 μg/ml anti-Fc antibody for 1 hour at room temperature, followed by Matrigel (Corning) for 20 minutes. Fc antibody coated dishes were incubated with 5 ng/μl of Fc fusion protein of each Notch ligand (Dll1, Dll4, Jag1 and Jag2) for 1 hour at room temperature. Individual myofibers isolated from EDL muscle were placed in serum-free DMEM and cultured in a CO₂ incubator for 48 hours. DAPT (10 UM) was used to inhibit γ-secretase activity in myofibers.

Co-culture experiments between myofibers and HEK293 cells were performed as follows. The isolated myofibers were cultured with HEK293 cells transfected with respective plasmids encoding GFP, Dll1-GFP and Dll4-GFP, in serum-free DMEM for 48 hours in a CO-incubator.

Transwell Permeable Supports (transwell membrane with 8 μm pores; Corning) were used for co-culture of myofibers and primary ECs isolated from muscle tissue. Individual myofibers and ECs were placed in plate wells (lower wells) and transwell inserts (upper wells), respectively, and cultured in serum-free DMEM in a CO₂ incubator for 48 hours. An anti-Dll4 neutralizing antibody (500 ng/ml) was added to the culture medium to block the Dll4 protein secreted from ECs, and a control antibody (500 ng/ml) was used as a control.

Cultured myofibers were captured using fluorescence microscopy and diameters were quantified from at least 15 individual myofibers using ImageJ (NIH).

### (12) Cell Sorting

Endothelial cells belonging to muscle tissue were freshly obtained by cell sorting and their gene expression was evaluated. Mouse GAS muscle was minced and digested with 0.2% type II collagenase followed by red blood cell lysis. Mononuclear cells were incubated with PE-conjugated CD31 antibody for 30 minutes at 4° C. CD31⁺ cells were sorted by S3e cell sorter (Bio Rad). Debris and dead cells were excluded by forward scatter and side scatter. Sorted cells were harvested into microtubes using ISOGEN II (Nippon Gene).

### (13) RNA Extraction and Quantitative Reverse Transcription PCR

Total RNA was extracted from muscle tissue, isolated myofibers, cultured cells, and freshly sorted cells using ISOGEN II and RNeasy Kit (Qiagen) according to the manufacturer's instructions. CDNA was prepared using the ReverTra Ace kit with genomic DNA remover (TOYOBO). Quantitative PCR (qPCR) was performed using THUNDERBIRD SYBR qPCR mix (TOYOBO) and real-time PCR detection system (CFX96; Bio Rad). The following primer sequences were used: TATA box binding protein (Tbp) as standard, and Pax7, Notch1, Notch2, Rbpj, Hes1, Dll1, Dll4, Jag1, Jag2, Ccng2, Cdkn1b, Rb12, Bnip3, MuRF1, Atrogin1, Fbxo31, and MUSA1.

### (14) RNA Sequencing

Total RNA was obtained from GAS muscle using ISOGEN II and the RNeasy kit. Library preparation and RNA sequencing were commissioned from Novogene (Beijing).

### (15) Immunofluorescence Method

Isolated PLA or TA muscles were immediately frozen in liquid nitrogen-cooled 2-methylbutane and stored at −80° C. until analysis. Frozen tissues were sliced at 10 μm thickness using a cryostat and immunohistochemical analysis of frozen sections was performed. Immunocytochemical analysis of satellite cells and isolated myofibers was performed according to the inventor's previous report (Ono et al., Cell Report 10, 1135-1148 (2015)). Samples were incubated with the primary antibody overnight at 4° C. Then, it was fixed with 4% paraformaldehyde (PFA)/PBS solution and permeabilized/blocked with PBS containing 0.3% TritonX-100 and 5% normal goat serum for 30 minutes at room temperature. Immunostained specimens were visualized using Alexa Fluor conjugated secondary antibodies and observed using fluorescence microscopy. Image-J (NIH) was used to quantify myofiber CSA from at least 500 myofibers and count the number of satellite cells per myofiber from at least 15 myofibers.

Whole-mount staining of muscle tissue was performed for vascular staining. Anesthetized mice were perfused with 4% PFA/PBS solution and GAS muscles were carefully harvested. The excised tissue was sliced to a thickness of 1 mm using a sharp razor and soaked overnight at 4° C. in a 1% bovine serum albumin/0.1% PBST blocking solution. Blocked tissues were incubated overnight at 4° C. with anti-CD31 and anti-Dll4 antibodies as primary antibodies. Tissues were washed three times with PBST and stained with appropriate species-specific secondary antibodies and DAPI (nuclear stain) overnight at 4° C. Muscles were again washed five times with PBST and analyzed using a confocal fluorescence microscope.

### (16) Transmission Electron Microscope (TEM)

Under deep anesthesia with inhaled isoflurane, mice were fixed by perfusion through the ascending aorta with 2% PFA and 2.5% glutaraldehyde in 0.1 M phosphate buffer. GAS muscles were trimmed into cubes (1 mm³) and post-fixed in 0.1 M phosphate buffer containing 1% OₛO₄ for 1 hour on ice, then, embedded in epoxy resin, block-stained with 1.5% uranium acetate, dehydrated in ethanol, infiltrated with propylene oxide, and embedded in araldite. Ultrathin sections (60-70 nm thick) were cut using an ultramicrotome and stained with 1.5% uranium acetate and lead citrate. Images were taken using a TEM (Hitachi H7700).

### (17) Correlated Confocal Scanning Laser Microscopy (CSLM)-Electron Microscopy (EM)

Under deep anesthesia with inhaled isoflurane, mice were fixed by perfusion through the ascending aorta with 4% PFA and 0.1% glutaraldehyde in 0.1 M phosphate buffer. Serial 70 μm-thick sections were cut from GAS muscle using a vibrating microtome (Dosaka TTK-3000) and observed with correlated CSLM-EM. Specifically, sections were subjected to rapid freezing and thawing in liquid nitrogen vapor followed by incubation at 4° C. for 7 days with an anti-Dll4 antibody (1:200), an anti-dystrophin antibody (1:300), and an anti-CD31 antibody (1:250), then, incubated overnight with biotinylated donkey anti-rat IgG (1:250). The sections were then incubated overnight with Alexa 488-conjugated anti-mouse IgG (1:200), rhodamine red-conjugated donkey anti-rabbit IgG (1:250), and Alexa 647-conjugated streptavidin (1:500). Longer incubation periods with primary and secondary antibodies were performed to improve antibody penetration into deeper sections of 70 μm thick sections. After acquiring high-resolution images using confocal laser scanning optical microscopy, the fluorescence signal of Dll4 was converted into the diaminobenzidine tetrahydrochloride (DAB) signal by overnight incubation with a mouse peroxidase-antiperoxidase complex (1:500), followed by a DAB reaction and post-fixation with 1% OₛO₄ and 1.5% uranium acetate. Sections were dehydrated and plate-embedded in epoxy resin. Ultrathin sections were cut using an ultramicrotome and then stained with uranium acetate and lead citrate. Images were taken using a TEM (Hitachi H7700).

### (18) Protein Extraction and Immunoblotting

Total protein lysates were obtained from cultured satellite cells or satellite cell-derived myotubes after exposure to RIPA buffer (FUJIFILM-Wako). The protein concentration in each sample was quantified using the Pierce BCA Protein Assay Kit (ThermoFisher). To quantify protein secretion into the medium during EC culture, the culture medium was concentrated using Amicon Ultra (Merck). Primary antibodies were diluted in CanGetSignal solution A (TOYOBO) and incubated overnight at 4° C. with membranes containing electrophoretically migrated protein bands. Membranes were then washed, incubated with horseradish peroxidase (HRP)-conjugated secondary antibody diluted in CanGetSignal solution B (TOYOBO) for 1 hour at room temperature, and visualized using chemiluminescence method.

### (19) Statistics

All statistical analyzes were performed using GraphPad Prism 8. Student's unpaired t-test was used for two-condition statistical comparisons. For comparisons of two or more groups, data were analyzed by one-way ANOVA followed by Dunnett's multiple comparison post hoc test, or two-way ANOVA followed by Tukey's or Sidak's multiple comparison post hoc test. P values of <0.05 (*), <0.01 (**), <0.001 (***) were considered statistically significant. All data represent the mean±standard error (SEM). n.s. indicates that the results were not statistically significant.

### (Example 1) Muscle Atrophy Induced by Notch2

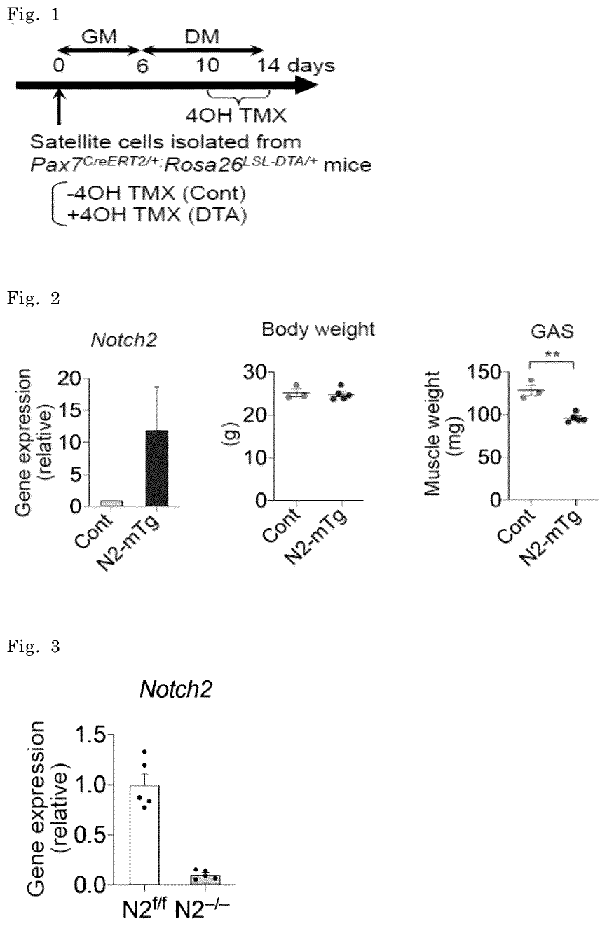

The present inventors have reported that satellite cells (skeletal muscle stem cells) express Notch1 and Notch2, which coordinately maintain the quiescent and undifferentiated states of satellite cells in adult mice (Non-patent Reference 2). Therefore, it was confirmed that Notch2 is expressed not only in undifferentiated mononuclear satellite cells but also in terminally differentiated multinucleated myofibers (myofibers). Specifically, satellite cells were isolated from the EDL muscle of Pax7^{creERTC/+}; Rosa26^{LSL-DTA/+} mice according to the method shown in FIG. 1, and cultured in myoblast growth medium (GM), then, the medium was switched to myoblast differentiation medium (DM) and treated with 4OH MTX to deplete undifferentiated satellite cells. Then, expression of Notch2 was measured by PCR analysis and Western blotting.

Next, to investigate the influences of constitutive expression of the active form of Notch2 in myofibers in vivo, HSA^{rtTA}; TR^{Cre} and Rosa26^{LSL-N2ICD} (N2 ICD-mTg) mice were prepared. In this mouse, the intracellular domain of Notch2 (N2ICD) is induced in a myofiber-specific manner by doxycycline (Dox). Tg and control mice were administered Dox (2 mg/ml in drinking water) for 3 weeks before Notch2 expression, body weight and muscle mass were measured. N2ICD-mTg mice showed no difference in body weight, but a significant decrease in muscle mass compared to controls. The results are shown in FIG. 2. This suggested that forced expression of N2ICD induces muscle atrophy.

### (Example 2) Test Using Muscle Atrophy Mouse Model

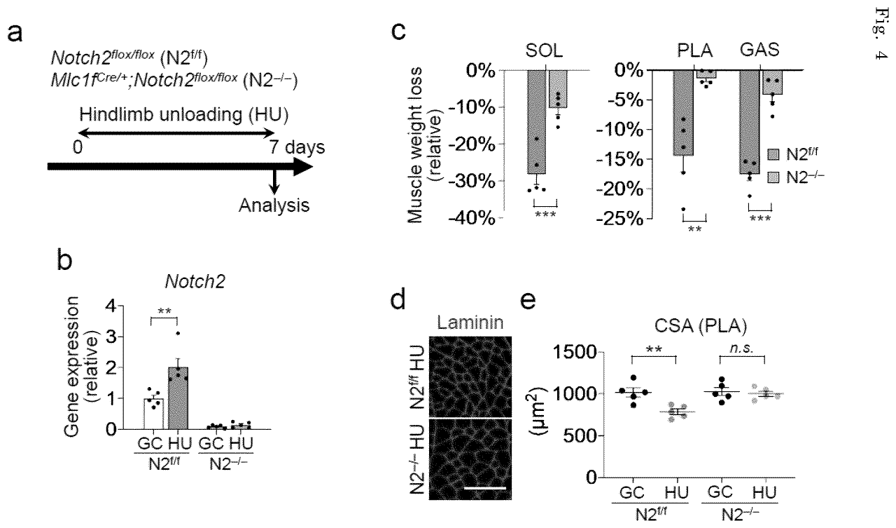

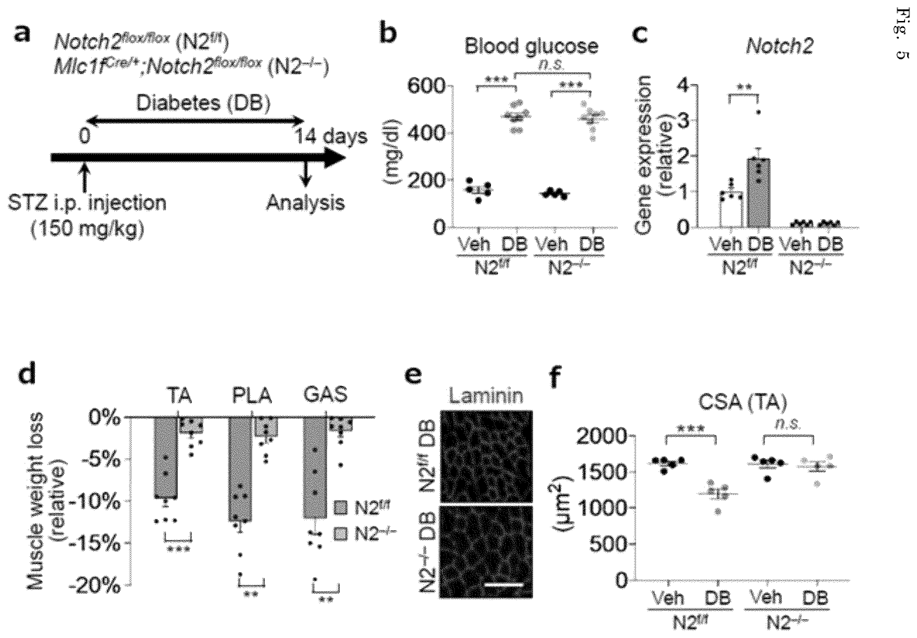

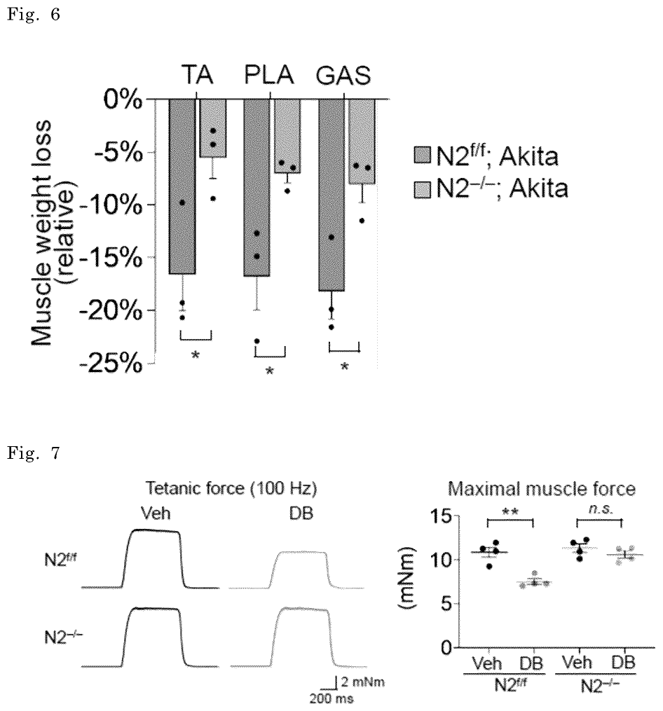

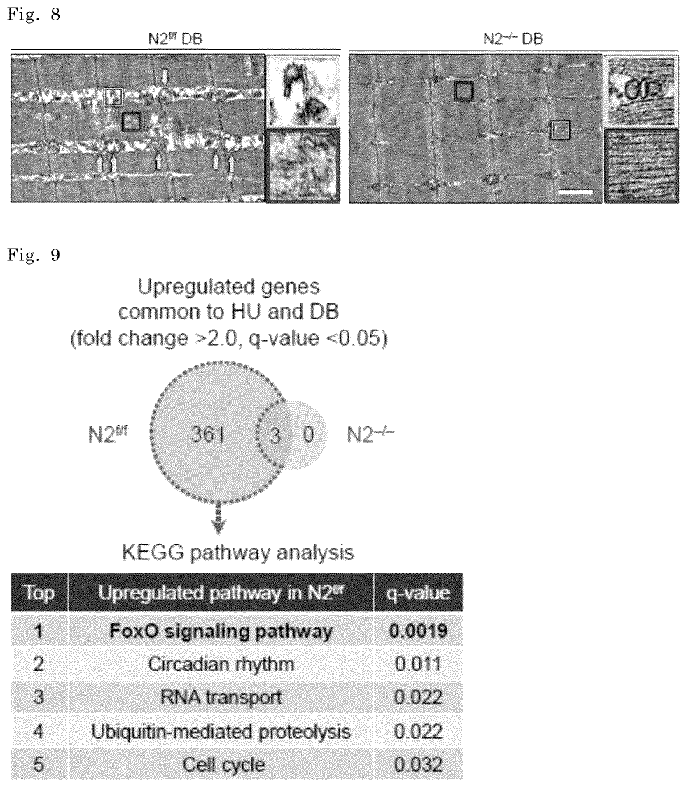

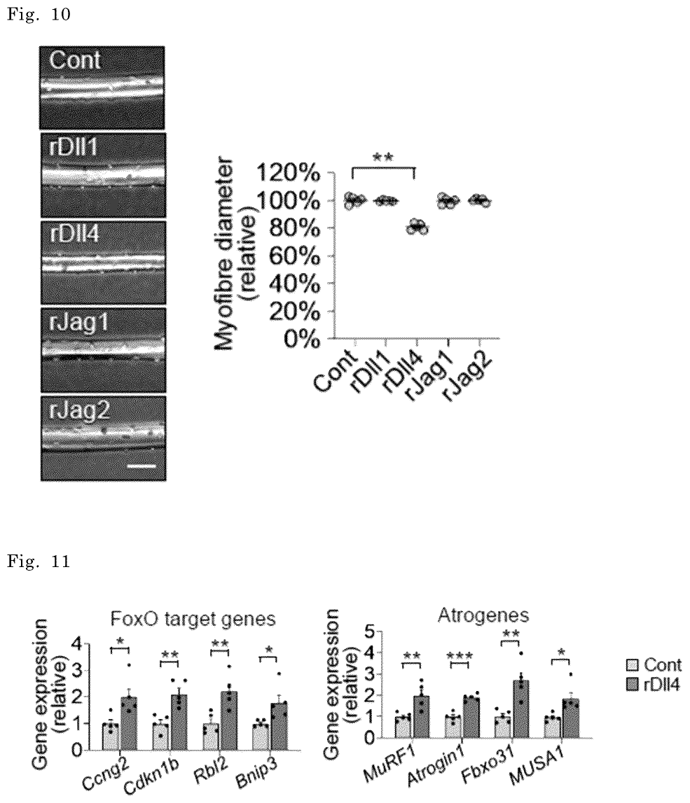

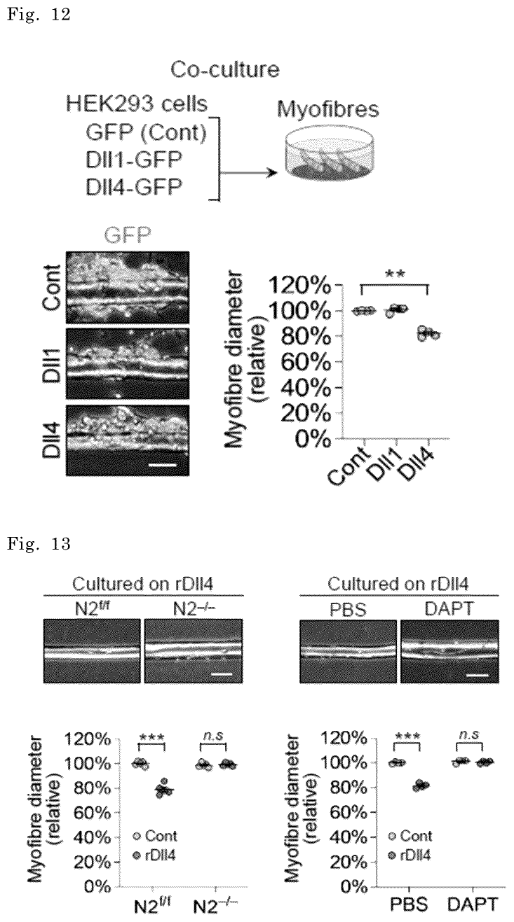

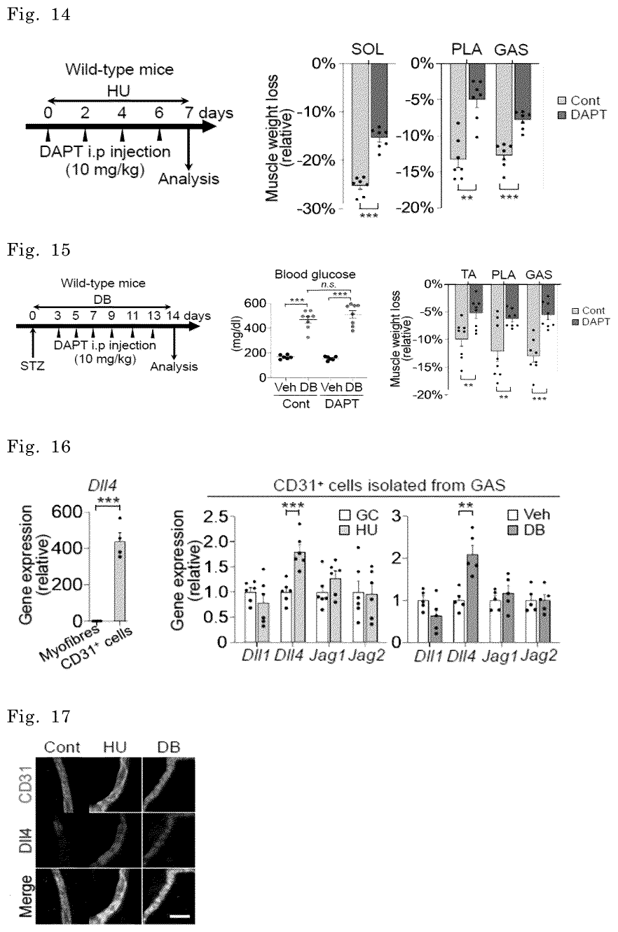

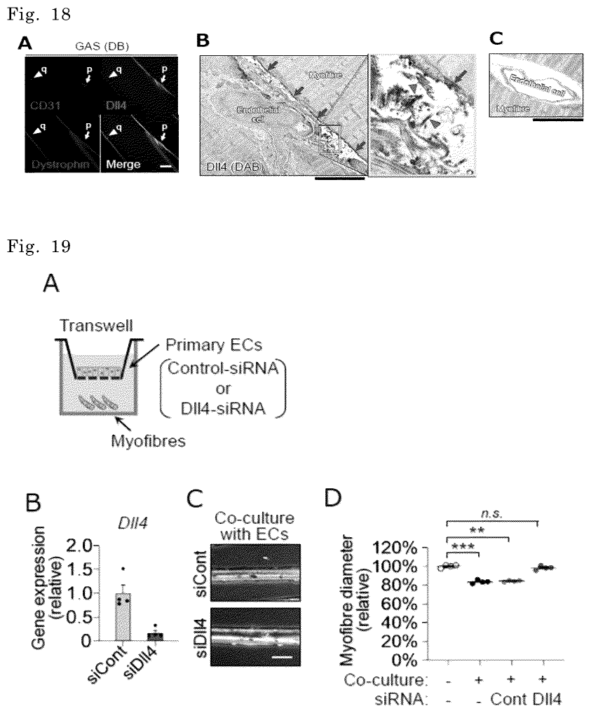

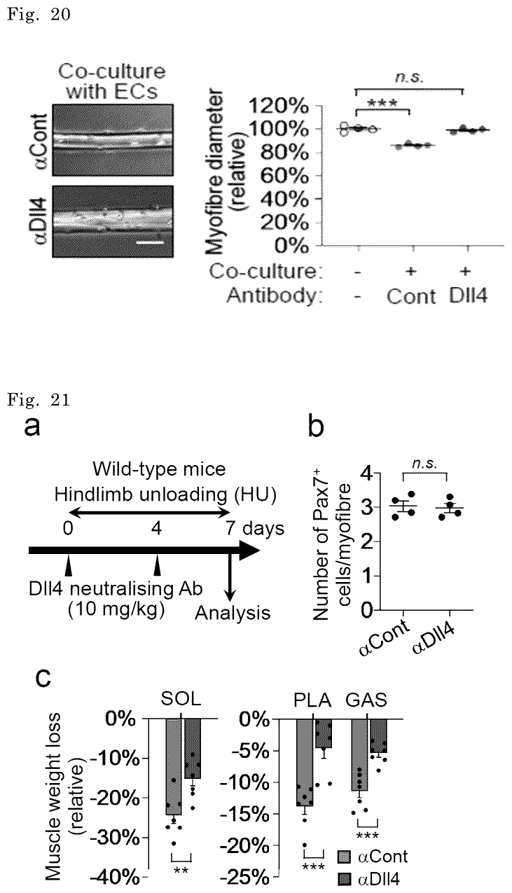

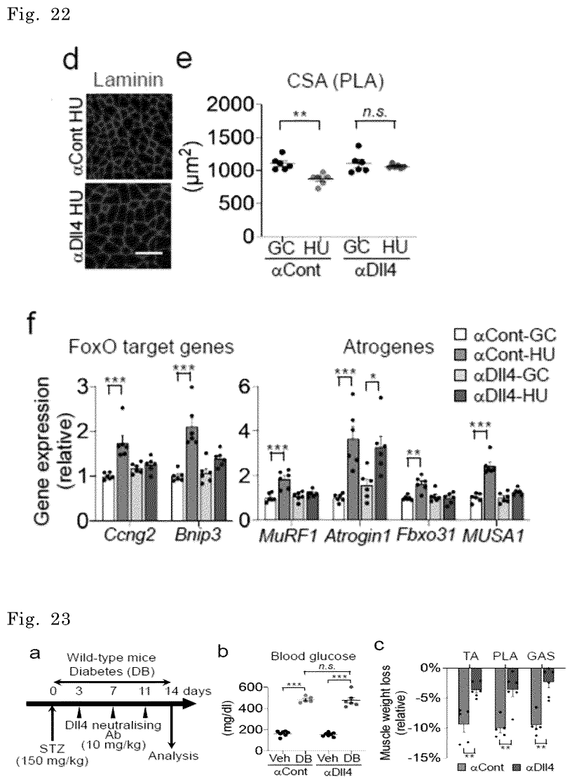

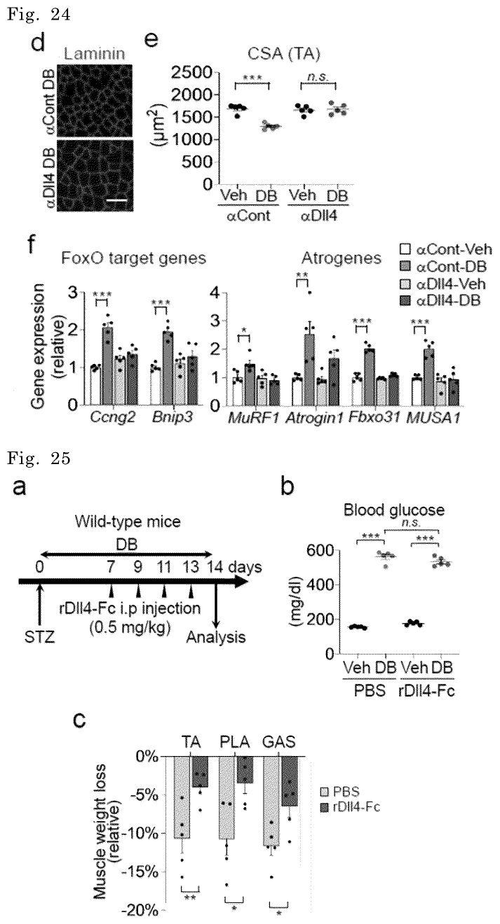

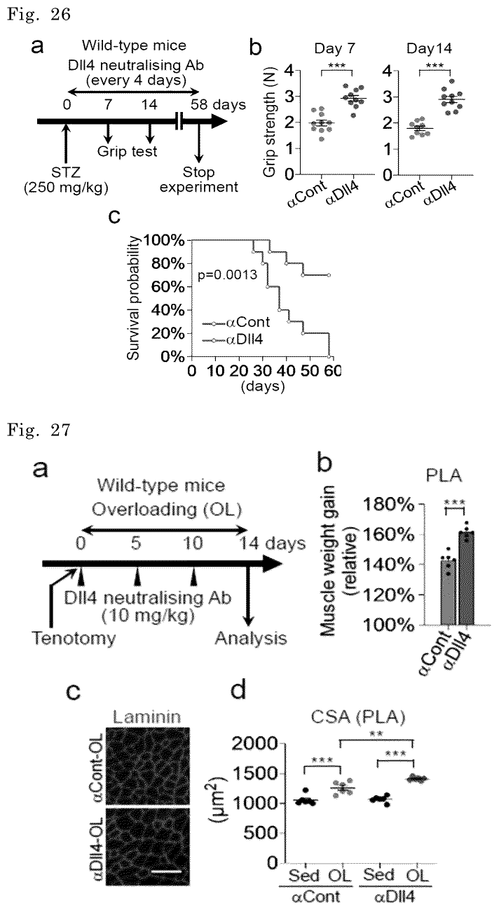

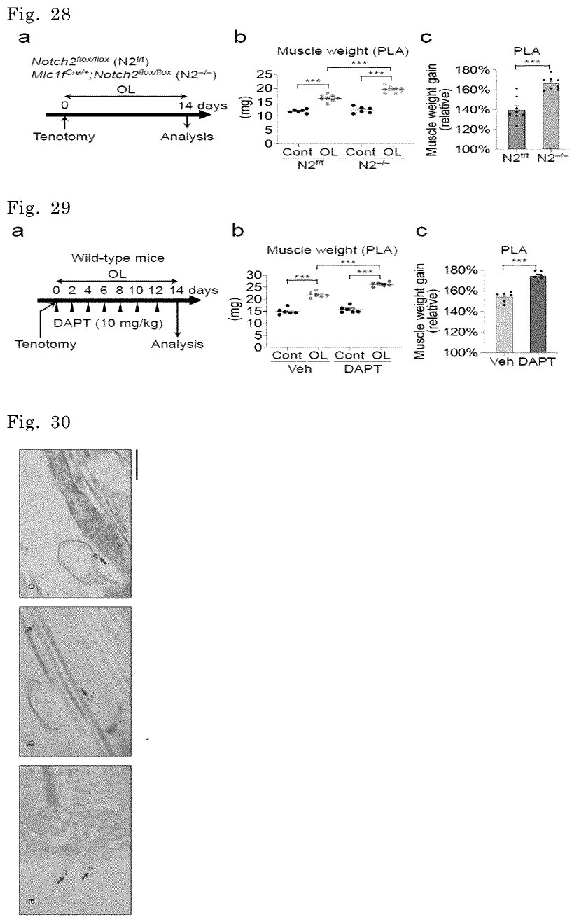

To investigate the role of Notch2 in myofibers, myosin light chain 1f promoter-driven Cre recombinase-expressing (Mlc^{Cre/+}) mice in which Notch2 expression is genetically ablated by Mlc1f^{Cre} (Mlc^{Cre/+}; N2^{f/f}, N2^{-/-}) were crossed with Notch2^{f/f} (N2^{f/f}) mice to generate myofiber-specific Notch2-deficient (N2^{-/-}) mice. As shown in FIG. 3, Notch2-deficient mice (N2^{-/-}) had decreased Notch2 expression, whereas Notch2-deficient mice grew normally and did not display an abnormal phenotype in terms of body weight, muscle weight, or number of myonuclei or Pax7⁺ satellite cells per extensor digitorum longus (EDL) myofiber.

Two different models of muscle atrophy, mechanical unloading (disuse) muscle atrophy and metabolic disorder (diabetes)-induced muscle atrophy, were used to test the influence by Notch2 inactivation. Mice with normal Notch2 expression (N2^{f/f}) and Notch2-deficient mice (N2^{-/-}) were used. As shown in FIG. 4, mechanical unloading was performed by hindlimb unloading (HU) for 7 days. In N2^{f/f} mice, HU increased Notch2 gene expression in plantar (PLA) myofibers compared to ground control (GC). It was found that HU reduced muscle mass in the soleus (SOL), PLA, and gastrocnemius (GAS) muscles of N2^{f/f} mice, whereas muscle mass in N2^{-/-} mice was mostly unaffected. Immunohistochemical analysis demonstrated that the cross-sectional area (CSA) of the PLA muscle was unchanged in the HU condition. Also, as shown in d, the thickness of individual myofibers was big for N2^{-/-} HU. This indicates that Notch2 expressed by myofibers is required to trigger mechanical unloading-induced muscle atrophy.

Diabetes (DB) causes numerous metabolic abnormalities, including persistent hyperglycemia, and is a high risk factor for muscle weakness. A streptozotocin (STZ)-induced DB mouse model was used that induces hyperglycemia by depleting pancreatic β-cells. Mice were injected intraperitoneally with STZ (150 mg/kg body weight) or vehicle (Veh) and analyzed 14 days later. N2^{f/f} and N2^{-/-} mice exhibited comparable hyperglycemia after STZ injection, and Notch2 gene expression was upregulated in EDL myofibers of DB and HU mice. Muscle mass of TA, PLA, and GAS muscles and CSA of TA muscles were markedly decreased in N2^{f/f} DB mice, whereas not decreased in N2^{-/-} DB mice. In addition, the thickness of individual myofibers was large in N2^{-/-} DB mice (FIG. 5e). The results are shown in FIG. 5.

### (Example 3) Test with Type I Diabetes Mouse Model (Akita Mouse Model)

Using a type I diabetes mouse model (Akita mouse model) in which Notch2 deletion was introduced, an influence of Notch2 on muscle atrophy was tested. As a result, it was demonstrated that muscle atrophy was reduced in the tibialis anterior (TA), PLA, and GAS in the Akita mouse model as well. The results are shown in FIG. 6.

### (Example 4) Comparison of N2^{f/f} DB Mice and N2^{-/-} DB Mice

When comparing muscle strength in the tibialis anterior (TA), TA force generation was markedly reduced in N2^{f/f} DB mice, whereas not decreased in N2^{-/-} DB mice, as shown in FIG. 7.

As a result of transmission electron microscopy (TEM) analysis for each mouse, abnormal triads and swollen mitochondria were observed in muscle of N2^{f/f} DB mice, while not observed in N2^{-/-} DB mice, as shown in FIG. 8.

These results demonstrate that Notch2 is required for loss of muscle mass and function due to mechanical unloading or dysmetabolic induction.

### (Example 5) Genetic Analysis in Muscle Under HU and DB Conditions

Under both HU and DB conditions, transcriptome analysis by RNA sequencing was performed for N2^{f/f} DB mice and N2^{-/-} DB mice, respectively. As a result, the expression of many genes was significantly altered in the muscle of N2^{f/f} DB mice, while few were statistically significantly altered in N2^{-/-} DB mice. This indicates that Notch2 primarily regulates gene expression dynamics in muscle induced by mechanical unloading and metabolic abnormalities. Under both HU and DB conditions, only eight genes were commonly identified as differentially expressed genes (DEGs) compared to each control.

Then, Kyoto Encyclopedia of Genes and Genomes (KEGG) pathway analysis was conducted using the common DEG under HU and DB conditions using N2^{f/f} mice, as a result, it was found that the FoxO signaling pathway was associated with the top DEG, as shown in FIG. 9. Analysis of the expression of FoxO target genes and atrogenes genes that induce muscle atrogenes showed that the expression of FoxO target genes such as Ccng2, Cdkn1b, Rb12 and Bnip318 was upregulated, as well as ubiquitin ligands such as MuRF1, Atrogin1, Fbxo31 and MUSA1, which are known as atrogenes that induce muscle atrogenes, in muscles of N2^{f/f} mice under HU and DB conditions. Conversely, these genes were not changed in muscle of N2^{-/-} mice.

However, myofiber-specific RBP-J-deficient mice and Hes1-deficient mice were not resistant to HU- and DB-induced muscle atrophy. Ablation of RBP-J and overexpression of N2ICD in myofibers decreased muscle mass, suggesting that forced activation of Notch2 may induce muscle atrophy irrespective of RBP-J. This indicated that unlike satellite cells regulated by the canonical Notch-RBP-J-Hes/Hey pathway, the canonical Notch-RBP-J signaling pathway is not involved in Notch2-mediated muscle atrophy. Taken together, it was shown that Notch2 acts as a key mediator of muscle atrophy in both mechanically and metabolically abnormal conditions, since Notch2 governs atrophic transcriptional activity in an RBP-J-independent manner.

### (Example 6) Investigation of Molecules that Regulate Notch2-Mediated Muscle Atrophy

It was investigated which of the five known Notch ligands activate Notch2 in myofibers. When the expression of each ligand in adult muscle tissue was measured, it was found that gene expression of four ligands (Jag1, Jag2, Dll1 and Dll4) excluding Dll3 could be detected in adult muscle tissue.

Subsequently, the actions of Jag1, Jag2, Dll1, and Dll4 on myofibers were evaluated. Using tissue culture plates coated on the bottom with these four recombinant proteins, myofibers freshly isolated from mouse EDL muscle were stimulated with the respective proteins in serum-free DMEM for 48 hours. The results are shown in FIG. 10. Myofiber atrophy was induced by Dll4 treatment alone, but not by other ligands. Gene expression analysis after 24 hours of culture on plates coated with Dll4 recombinant protein revealed that Dll4-stimulated myofibers upregulated FoxO target genes and atrogen, as shown in FIG. 11. These results were consistent with an in vivo atrophy model.

### (Example 7) Investigation of Induction of Muscle Atrophy Via Cell-to-Cell Contact

To investigate whether Notch ligands through cell-to-cell contact can induce myofiber atrophy, HEK293 cells were transfected with either Dll1 or Dll4 expression plasmid vectors and co-cultured with isolated myofibers for 48 hours, and its expression in myofibers was measured. FIG. 12 shows the result of co-culturing EDL myofibers with HEK293 cells transfected with plasmids encoding Dll1-GFP, Dll4-GFP, or GFP (Cont.), respectively. Co-culture with Dll1-expressing HEK293 cells had no change. In addition, compared to co-culture with Dll1-expressing HEK293 cells, co-culture with Dll4-expressing HEK293 cells significantly decreased the size of myofibers, making it more apparent that Dll4 is the specific ligand that induces myofiber atrophy.

### (Example 7) Investigation of γ-Secretase Inhibitor Against Dll4-Induced Muscle Atrophy

Notch, which is a transmembrane receptor, has its transmembrane domain cleaved by γ-secretase, translocates its intracellular domain to the nucleus, and functions as a transcription factor. Therefore, DAPT, a γ-secretase inhibitor, was used to test the influence of γ-secretase activity on Dll4-induced myofiber atrophy. EDL myofibers derived from N2^{f/f} mice were cultured for 48 hours on a plate coated previously with recombinant Dll4 protein using a medium containing a γ-secretase inhibitor DAPT (10 μM) or PBS. The results are shown in FIG. 13. Both N2^{-/-} myofibers and γ-secretase inhibitor (DAPT)-treated myofibers were resistant to Dll4 stimulation. This demonstrated that Dll4-induced muscle atrophy was mediated by Notch2 and γ-secretase activity.

Then, the effects of γ-secretase inhibitor in two models of muscle atrophy, mechanical unloading muscle atrophy (HU) and metabolic disorder (diabetes)-induced muscle atrophy (DB), were tested by intraperitoneal injections of the γ-secretase inhibitor DAPT (10 mg/kg body weight) or corn oil (Cont) to wild-type mice every other day, during the HU period or DB induced period. FIG. 14 shows the results using the mechanical unloading muscle atrophy (HU) model, and FIG. 15 shows the results using the metabolic disorder (diabetes)-induced muscle atrophy (DB) model. Treatment with DAPT was found to lead to significant suppression of muscle atrophy in both HU and DB models. These results indicate that the Dll4-Notch2 signaling axis induces muscle atrophy via γ-secretase activity.

### (Example 8) Identification of the Origin of Dll4

Identification of the origin of the Dll4 ligand was then carried out. To confirm the site where Dll4 ligand is expressing, expression of Dll4 ligand in CD31⁺ endothelial cells (endothelial cell marker CD31 positive EC) derived from adult muscle tissue was measured and compared to freshly isolated myofibers. The results are shown in the left figure of FIG. 16. As a result, the Dll4 gene was highly expressed about 400-fold in CD31⁺ EC. In addition, using wild-type mice, expression of Notch ligand (Dll1, Dll4, Jag1 and Jag2) in CD31⁺ endothelial cells derived from GAS muscle was measured under both HU and DB conditions. The results are shown in the right figure of FIG. 16. Dll4 was found to be upregulated in both HU and DB conditions, but the other ligands were not. Immunohistochemical analysis also demonstrated upregulation of Dll4. The results are shown in FIG. 17. In addition, immunoelectron microscopic analysis was performed. The results are shown in FIG. 18. As shown in FIG. 18A, Dll4 was found to be distributed not only on the surface of ECs but also on collagen-like fibers in the interstitial space between ECs and myofibers of diabetic muscle tissue. Furthermore, as shown in FIGS. 18B and C, Dll4 was also enriched on the surface of myofibers.

These results suggest that in muscle tissue under DB conditions, EC upregulates and secretes Dll4 into the interstices, and the secreted Dll4 would selectively activate Notch2 receptors expressed on the surface of myofibers.

Expression of Dll4 in EC was tested by immunoblot analysis. EC isolated from muscle tissue was cultured under the low glucose condition (5 mM) similar to healthy blood level (90 mg/dl) and under the high glucose condition (25 mM) mimicking the level of diabetic hyperglycemia in blood (450 mg/dl), and Dll4 in the supernatant was measured, as a result, higher expression levels were shown under the high glucose condition (25 mM) than under the low glucose condition (5 mM).

### (Example 9) Investigation of Dll4 Expression Inhibition

The function of EC-secreted Dll4 in myofiber atrophy was evaluated as follows. A co-culture system of primary ECs isolated from myofibers and myofibers was prepared using Transwell, as shown in FIG. 19A. EC-derived secretions pass through the upper chamber to reach myofibers laid on the bottom surface. Primary ECs were transfected with control siRNA (siCont) or siRNA against Dll4 (siDll4) one day before co-culture with myofibers and co-culture was performed. Expression of Dll4 in EC transfected with siCont or siDll4 is shown in FIG. 19B. As shown in FIGS. 19C and D, in this system, co-culture with EC resulted in a marked decrease in myofiber size, and it was found that this myofiber atrophy could be completely rescued by siRNA-mediated knockdown of Dll4 in EC.

### (Example 10) Investigation of Inhibition of Dll4

Using the same co-culture system as in Example 9, the effect of the anti-Dll4 neutralizing antibody was tested. The results are shown in FIG. 20. Treatment with anti-Dll4 neutralizing antibody (αDll4) (500 ng/ml) completely suppressed myofiber atrophy in the co-culture system.

These results demonstrate that EC-derived Dll4 can activate Notch2 in myofibers and induce muscle atrophy without direct cell-to-cell contact.

### (Example 11) Suppression of Muscle Atrophy Using Anti-Dll4 Antibody (HU Model)

Using a neutralizing antibody against Dll4 protein, the effect of suppressing muscle atrophy was examined as follows. Muscle atrophy was induced in wild-type mice under HU conditions. Mice were injected intraperitoneally twice with anti-Dll4 antibody (αDll4) (10 mg/kg body weight) or control antibody (αCont) during the 7-day period of HU (on days 0 and 4). Seven days after the first dose of antibody, mice were euthanized and analyzed. The results are shown in FIGS. 21 and 22. It was found that αDll4 treatment dramatically reduced the HU-induced loss of muscle mass and CSA in SOL, PLA, and GAS muscles, although it did not affect the number of satellite cells per myofiber. This demonstrated that anti-Dll4 antibody can be used to suppress muscle atrophy. As shown in d, the thickness of individual myofibers was greater in αDll4 HU. As a result of genetic analysis, the expression levels of FoxO target genes and muscle atrophy-causing genes (atrogenes) excluding Atrogin1 were also suppressed by αDll4.

### (Example 12) Suppression of Muscle Atrophy Using Anti-Dll4 Antibody (DB Model)

Using a wild-type mouse DB-induced muscle atrophy model, the muscle atrophy suppression effect of a neutralizing antibody against Dll4 protein was examined. After injection of STZ (150 mg/kg body weight; DB) or solvent (veh; citrate buffer) into mice, αDll4 (10 mg/kg body weight) or control antibody (αCont) was administered intraperitoneally three times over 2 weeks (on days 3, 7, and 11 after STZ administration). Analysis was performed 14 days after STZ administration. The results are shown in FIGS. 23 and 24. Administration of αDll4 showed a strong protective effect against DB-induced muscle atrophy without affecting blood glucose levels. As shown in FIG. 24d, the thickness of individual myofibers was greater in αDll4 DB.

### (Example 13) Suppression of Muscle Atrophy Using Soluble Recombinant Anti-Dll4 Protein

Using a wild-type mouse DB-induced muscle atrophy model, the muscle atrophy suppression effect by soluble recombinant Dll4-Fc protein, which prevents binding of endogenous Dll4 to Notch receptors, was examined. After intraperitoneal injection of STZ (150 mg/kg body weight; DB) or solvent (Veh; citrate buffer) to wild-type mice, soluble Dll4-Fc recombinant protein (rDll4-Fc) (0.5 mg/kg) was administered four times (on days 7, 9, 11, and 13) over a period of 14 days. Analysis was performed 14 days after administration of STZ. PBS was used as a control. The results are shown in FIG. 25. Administration of soluble recombinant Dll4-Fc protein was demonstrated to attenuate DB-induced muscle atrophy without affecting blood glucose levels.

It was demonstrated from these results that a preventive/therapeutic drug that targets the Dll4 protein can serve as a preventive/therapeutic agent for muscle weakness due to disuse and diabetic conditions. However, when endothelial cell-specific Dll4 knockout mice were generated and analyzed, muscle mass was slightly reduced and no protective effect against HU-induced muscle atrophy was observed, suggesting that appropriate concentrations of Dll4 in endothelial cells is essential for maintaining tissue homeostasis.

### (Example 14) Effect of Anti-Dll4 Antibody on Severe Hyperglycemia

The effect of anti-Dll4 antibody (αDll4) on mortality in STZ-induced severe hyperglycemia in mice was evaluated. Wild-type mice were administered a very high dose of STZ (250 mg/kg) to induce severe hyperglycemia. Twenty STZ-injected wild-type mice were divided into two groups treated with either αDll4 or αCont (each dosed at 10 mg/kg body weight). Antibodies were administered intraperitoneally every 4 days and continued until mice became weak or died (up to 58 days). The results are shown in FIG. 26. Both the αDll4 and αCon groups exhibited severe hyperglycemia (blood glucose<600 mg/dl) after STZ infusion. Death or euthanasia were shown as Kaplan-Meier plots over time. While αCon-treated mice had a short lifespan (approximately 58 days), αDll4-treated mice had a significantly higher survival rate (70% survival rate) and increased muscle strength. This result indicates that attenuation of αDll4 activity exerts a protective effect against systemic homeostasis disruption from hyperglycemia.

### (Example 15) Muscle Hypertrophy Effect by Dll4 Inhibition

It was examined whether attenuation of the Dll4-Notch2 signaling axis promotes muscle hypertrophy due to mechanical overload. Using wild-type mice, muscle hypertrophy was induced by mechanical overloading (OL). PLA muscle hypertrophy was induced by synergistic ablation (tenotomy) of the SOL and GAS muscles of both legs. As a control, synergistic muscle non-tenotomy mice were used. After tenotomy, αDll4 (10 mg/kg body weight) or αCont was administered intraperitoneally twice over 2 weeks (on days 5 and 10 after resection) and analyzed on day 14. The results are shown in FIG. 27. Hypertrophic efficiency, as measured by muscle mass and cross-sectional area of PLA muscle, was significantly higher in αDll4-treated mice compared to αCon-treated mice. In addition, as shown in c, the thickness of individual myofibers was greater in αDll4 OL.

Using a mechanical overload-induced muscle hypertrophy model, the test was performed in N2^{-/-} mice and DAPT-treated mice. The results are shown in FIGS. 28 and 29. In both cases, muscle hypertrophy was exhibited more readily compared to control mice.

The above results indicated that the endothelial Dll4-myofiber Notch2 axis is important not only for inducing muscle atrophy but also for suppressing muscle hypertrophy.

### (Example 16) Distribution of Intramuscular Dll4

Immunoelectron microscopy analysis demonstrated the distribution of Dll4 in the muscle. Using the STZ-induced DB mouse model, 60-micrometer-thick vibratome sections were made from harvested GAS muscle. Sections were treated overnight for immunohistochemical staining using a primary antibody against Dll4 (1:50) and then incubated overnight with 10 nm-nanogold goat anti-mouse IgG (1:50, CRL). Sections were then post-fixed with 1% OsO₄ aqueous solution, bulk stained with 1.5% uranyl acetate, dehydrated and flat-embedded in epoxy resin. Ultrathin sections were stained with uranyl acetate and lead citrate and examined with a transmission electron microscope (H-7700, Hitachi). The results are shown in FIG. 30. Dll4 was detected to be distributed in the interstitial space between ECs and myofibers (particularly associated with collagen-like fibers and extracellular vesicles) and on the surface of ECs and myofibers. Therefore, it was suggested that in muscle tissue under DB conditions, ECs suppress Dll4, secrete it into the interstitial space, and selectively activate Notch2 receptors expressed on the surface of myofibers.

The foregoing merely illustrates objects and subjects of the present invention and is not intended to be limiting the accompanying Claims. Without departing from the accompanying Claims, various modifications and alterations to the described embodiments will be apparent to those skilled in the art in view of the teachings herein.

### INDUSTRIAL APPLICABILITY

The present invention provides a preventive or therapeutic agent for muscular atrophy based on a new mechanism of action. The composition of the present invention is useful for preventing, treating or improving muscle atrophy or muscle mass loss.

This application is based on Japanese Patent Application No. 2021-56481 (filing date: Mar. 30, 2021) filed in Japan, the entire contents of which are incorporated herein.

1-11. (canceled)

12. A method for preventing or treating a subject suffering from muscle atrophy by administering to the subject a therapeutically effective amount of a Dll4 function inhibitor,
wherein the Dll4 function inhibitor is selected from the group consisting of a binding inhibitor between Dll4 and Notch2, a Dll4 expression inhibitor and a collagen binding inhibitor to Dll4.

13. The method according to claim 12, wherein the Dll4 is Dll4 expressed in endothelial cells of blood vessels present in muscle tissue.

14. The method according to claim 12, wherein the binding inhibitor is an anti-Dll4 antibody that inhibits binding of Dll4 to Notch2.

15. The method according to claim 12, wherein the Dll4 expression inhibitor is siRNA or shRNA that induces RNA interference with Dll4 mRNA.

16. The method according to claim 12, wherein the muscular atrophy is a neurogenic amyotrophic disease or a myogenic amyotrophic disease.

17. The method according to claim 16, wherein the muscular atrophy is a neurogenic amyotrophic disease selected from the group consisting of amyotrophic lateral sclerosis (ALS), myasthenia gravis, progressive muscular atrophy, spinal muscular atrophy, spinobulbar muscular atrophy, Guillain-Barré syndrome, and muscle atrophy due to physical spinal cord injury.

18. The method according to claim 16, wherein the muscular atrophy is a myogenic amyotrophic disease selected from the group consisting of age-related muscular atrophy called as sarcopenia, disuse muscular atrophy, muscular dystrophy, diabetic muscular atrophy, congenital myopathy, distal myopathy, steroid myopathy, drug-induced myopathy, rhabdomyolysis, muscle wasting disease, Charcot-Marie-Tooth disease, Lambert-Eaton syndrome, muscle atrophy induced by cachexia, and muscle atrophy associated with burns.

19. A preventive or therapeutic agent for muscular atrophy comprising a Dll4 function inhibitor as an active ingredient,
wherein the Dll4 function inhibitor is selected from the group consisting of a binding inhibitor between Dll4 and Notch2, a Dll4 expression inhibitor and a collagen binding inhibitor to Dll4.

20. The preventive or therapeutic agent for muscular atrophy according to claim 19, wherein the Dll4 is Dll4 expressed in endothelial cells of blood vessels present in muscle tissue.

21. The preventive or therapeutic agent for muscular atrophy according to claim 19, wherein the binding inhibitor is an anti-Dll4 antibody that inhibits binding of Dll4 to Notch2.

22. The preventive or therapeutic agent for muscular atrophy according to claim 19, wherein the Dll4 expression inhibitor is siRNA or shRNA that induces RNA interference with Dll4 mRNA.

23. The preventive or therapeutic agent for muscular atrophy according to claim 19, wherein the muscular atrophy is a neurogenic amyotrophic disease or a myogenic amyotrophic disease.

24. The preventive or therapeutic agent for muscular atrophy according to claim 23, wherein the muscular atrophy is a neurogenic amyotrophic disease selected from the group consisting of amyotrophic lateral sclerosis (ALS), myasthenia gravis, progressive muscular atrophy, spinal muscular atrophy, spinobulbar muscular atrophy, Guillain-Barré syndrome, and muscle atrophy due to physical spinal cord injury.

25. The preventive or therapeutic agent for muscular atrophy according to claim 23, wherein the muscular atrophy is a myogenic amyotrophic disease selected from the group consisting of age-related muscular atrophy called as sarcopenia, disuse muscular atrophy, muscular dystrophy, diabetic muscular atrophy, congenital myopathy, distal myopathy, steroid myopathy, drug-induced myopathy, rhabdomyolysis, muscle wasting disease, Charcot-Marie-Tooth disease, Lambert-Eaton syndrome, muscle atrophy induced by cachexia, and muscle atrophy associated with burns.
